(19) United States
(12) Patent Application Publication
(10) Pub. No.: US 2023/0250461 A1
(43) Pub. Date: Aug. 10, 2023

(54) Compositions and Methods for Selective Detection and/or Inhibition of Bacterial Pathogens in Microbial Communities

(71) Applicant: YALE UNIVERSITY, New Haven, CT (US)

(72) Inventors: Stavroula Hatzios, New Haven, CT (US); Alicia Decolli, New Haven, CT (US)

(21) Appl. No.: 18/003,125

(22) PCT Filed: Jun. 25, 2021
(86) PCT No.: PCT/US2021/039062

§ 371 Date: Dec. 22, 2022

Related U.S. Application Data
(60) Provisional application No. 63/044,426, filed on Jun. 26, 2020.

(51) Int. Cl. C12Q 1/04; C12Q 1/37

(52) U.S. Cl. CPC C12Q 1/04; C12Q 1/37

(57) ABSTRACT

The present disclosure relates, in one aspect, to the unexpected discovery that the native enzymatic machinery of pathogenic bacteria can be harnessed to develop biosensors and antimicrobials that target disease-causing microbes in the gut. The present approach relates in one aspect to the use of proteases for the design of these precision tools.

### CROSS-REFERENCE TO RELATED APPLICATIONS

This application claims priority under 35 U.S.C. § 119(e) to U.S. Provisional Patent Application No. 63/044,426, filed Jun. 26, 2020, the contents of which are incorporated herein by reference in its entirety.

### SEQUENCE LISTING

The ASCII text filed named “047162-7287WO1(01382)_Sequence Listing_ST25” created on Jun. 2, 2021, comprising 14.8 KB, is hereby incorporated by reference in its entirety.

### BACKGROUND

The rapid diagnosis and treatment of bacterial infections is critical for controlling the spread of disease. However, selectively detecting pathogenic bacteria in patient samples, and eliminating these microbes from the body, are complicated by native microbial communities in the host (i.e., the microbiota). For example, identifying pathogens in microbe-rich stool samples can require lengthy sample processing and culture methods to distinguish the disease-causing bacterium from the hundreds of other strains present in fecal matter. Similarly, broad-spectrum antibiotics that nonspecifically target the microbiota can promote the emergence of antibiotic-resistant bacteria and the development of severe chronic illnesses. Antibiotic exposure drives the evolution of resistance mechanisms that can be acquired by other microbes via horizontal gene transfer. In addition, the indiscriminate killing of beneficial microbes has been linked to a number of persistent ailments including autoimmune disorders, allergies, and asthma.

Consequently, there is a demand for technologies that can rapidly detect and/or selectively kill pathogenic bacteria within complex microbial communities. For that, it is necessary to identify biomarkers of pathogenic bacterial infections and to design biosensors that detect the biomarker of infection with high specificity and sensitivity. The present invention fulfills this need.

### SUMMARY OF THE DISCLOSURE

In one aspect, the present disclosure relates to a construct comprising a pathogenic bacterium protease propeptide conjugated to a detectable label. In certain embodiments, the detectable label comprises a fluorophore, chromophore, magnetic nanoparticle, or any other detectable label. In certain embodiments, the fluorophore comprises a solvatochromic label.

In certain embodiments, the solvatochromic label comprises one of the following:

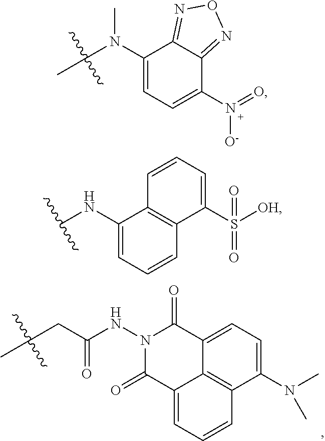

a charge transfer dye, an excited-state intramolecular proton transfer dye, and/or a merocyanine dye. In certain embodiments, the fluorophore comprises a TCC probe. In certain embodiments, the fluorophore comprises a conformation-sensitive fluorophore. In certain embodiments, the conformation-sensitive fluorophore is

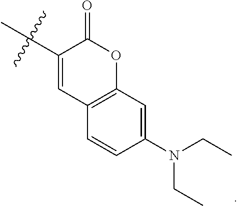 [SMILES: *c1cc2ccc(N(CC)CC)cc2oc1=O]

In certain embodiments, the detectable label emits a distinct signal (for example, a signal that is stronger or weaker in intensity, and/or a signal of distinct wavelength profile and/or wavelength maximum, as compared to the detectable label in the non-cleaved propeptide) once the propeptide is cleaved. In certain embodiments, the label comprises a fluorophore with FRET-based quenchers that are uncoupled by propeptide cleavage and/or a cleavage-activated chromophore. In certain embodiments, the propeptide is conjugated to the detectable label through a direct covalent bond. In certain embodiments, the propeptide is conjugated to the detectable label through a linker. In certain embodiments, the linker comprises from 1 to about 100 amino acids, or wherein the linker comprises from about 1 to about 50 residues selected from CH₂, CH₂CH₂, —C(═O)NH—, —C(═O)NCH₃—, CH(CH₃)CH₂, CH₂CH₂O, C(CH₃)CH₂O, and

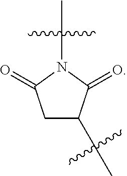 [SMILES: *C1CC(=O)N(*)C1=O]

In certain embodiments, the linker is

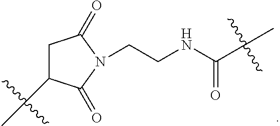 [SMILES: *C(=O)NCCN1C(=O)CC(*)C1=O]

In certain embodiments, the protease is a subtilisin. In certain embodiments, the propeptide is a I9 propeptide. In certain embodiments, the propeptide is a I9 propeptide for IvaP from Vibrio cholerae or a I9 propeptide for CspB of C. difficile. In certain embodiments, the propeptide is from Protease IV. In one embodiment, the propeptide is from Protease IV from Pseudomonas aeruginosa. In certain embodiments, the detectable label is attached to: (i) a residue of the propeptide that is present in the interface of the propeptide-protease complex once the propeptide binds to its corresponding protease; and/or (ii) a residue of the propeptide that is at a protease cleavage site.

In another aspect, the present disclosure relates to a method of identifying a pathogenic bacterium in a sample, the method comprising: contacting the construct described above (construct comprising a pathogenic bacterium protease propeptide conjugated to a detectable label) with the sample, wherein the bacterium produces the protease that binds to the propeptide, and monitoring a detectable signal for the label before and after the contacting, whereby a qualitative and/or quantitative change in the detectable signal upon contacting indicates that the pathogenic bacterium is present in the sample. In certain embodiments, the sample comprises another bacterium that does not produce the protease that binds to the propeptide. In certain embodiments, the detectable label comprises a fluorophore, chromophore, or any other detectable label.

In yet another aspect, the present disclosure relates to a construct comprising a pathogenic bacterium protease propeptide conjugated to an antimicrobial peptide (AMP) and/or a therapeutically active small molecule and/or a detectable label that emits a distinct detectable signal (for example, a signal that is stronger or weaker in intensity, and/or a signal of distinct wavelength profile and/or wavelength maximum, as compared to the detectable label in the non-cleaved propeptide) once the propeptide is cleaved. In certain embodiments, the propeptide is conjugated to the N-terminus of the AMP. In certain embodiments, the propeptide is conjugated to the AMP and/or therapeutically active small molecule and/or detectable label through a direct covalent bond. In certain embodiments, the propeptide is conjugated to the AMP and/or therapeutically active small molecule and/or detectable label through a linker. In certain embodiments, the linker comprises from 1 to about 100 amino acids, or wherein the linker comprises from about 1 to about 50 residues selected from CH₂, CH₂CH₂, —C(═O)NH—, —C(═O)NCH₃—, CH(CH₃)CH₂, CH₂CH₂O, C(CH₃)CH₂O, and

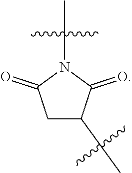 [SMILES: *C1CC(=O)N(*)C1=O]

In certain embodiments, the linker is

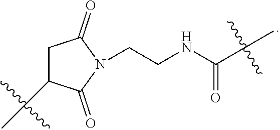 [SMILES: *C(=O)NCCN1C(=O)CC(*)C1=O]

In certain embodiments, the protease is a subtilisin. In certain embodiments, the propeptide is a I9 propeptide. In certain embodiments, the propeptide is a I9 propeptide for IvaP from Vibrio cholerae or a I9 propeptide for CspB of C. difficile. In certain embodiments, the propeptide is from Protease IV. In certain embodiments, the propeptide is from Protease IV from Pseudomonas aeruginosa.

In another aspect, the present disclosure relates to a method of killing a pathogenic bacterium, or reducing or preventing growth of a pathogenic bacterium, in a sample, the method comprising: contacting the construct described above (construct comprising a pathogenic bacterium protease propeptide conjugated to an AMP and/or a therapeutically active small molecule and/or a detectable label that emits a stronger detectable signal once the propeptide is cleaved) with the bacterium, wherein the bacterium produces the protease that binds to the propeptide; whereby the AMP is released from the construct upon the contacting. In certain embodiments, the method is performed in vivo in a subject infected by the pathogenic bacterium. In certain embodiments, the pathogenic bacterium is in the digestive tract, respiratory tract, urinary tract, and/or skin of the subject. In certain embodiments, non-pathogenic bacteria in the gut of the subject are not significantly killed, or wherein growth of non-pathogenic bacteria in the gut is not significantly reduced or prevented.

In yet another aspect, the present disclosure relates to a construct comprising a pathogenic bacterium protease propeptide conjugated to an electrophilic agent. In certain embodiments, the electrophilic agent is conjugated to the C-terminus of the propeptide and/or a mutated residue of the propeptide. In certain embodiments, the electrophilic agent comprises a fluorophosphate group. In certain embodiments, the propeptide is conjugated to the electrophilic agent through a direct covalent bond. In certain embodiments, the propeptide is conjugated to the electrophilic agent through a linker. In certain embodiments, the linker comprises from 1 to about 100 amino acids, or wherein the linker comprises from about 1 to about 50 residues selected from CH₂, CH₂CH₂, —C(═O)NH—, —C(═O)NCH₃—, CH(CH₃)CH₂, CH₂CH₂O, C(CH₃)CH₂O, and

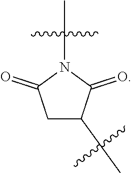 [SMILES: *C1CC(=O)N(*)C1=O]

In certain embodiments, the protease is a subtilisin. In certain embodiments, the propeptide is a I9 propeptide. In certain embodiments, the propeptide is a I9 propeptide for IvaP from Vibrio cholerae or a I9 propeptide for CspB of C. difficile. In certain embodiments, the propeptide is from Protease IV. In certain embodiments, the propeptide is from Protease IV from Pseudomonas aeruginosa.

In another aspect, the present disclosure relates to a method of inactivating a protease of a pathogenic bacterium, the method comprising contacting the construct described above (construct comprising a pathogenic bacterium protease propeptide conjugated to an electrophilic agent) with the bacterium, wherein the bacterium produces the protease that binds to the propeptide.

### BRIEF DESCRIPTION OF THE DRAWINGS

The following detailed description of illustrative embodiments of the disclosure will be better understood when read in conjunction with the appended drawings. For the purpose of illustrating the disclosure, exemplary embodiments are shown in the drawings. It should be understood, however, that the disclosure is not limited to the precise arrangements and instrumentalities of the embodiments shown in the drawings.

FIG. 1 illustrates an overview of chemical biology approach for detecting and/or killing pathogens using propeptide-based precision tools. Activity-based proteomics coupled with bioinformatic analyses can be used to identify auto-inhibitory propeptides that are cleaved by active proteases during infection. Site-specific conjugation of environment-sensitive dyes to purified propeptides allows for fluorescence detection of protease binding to its partner propeptide. Propeptide fusion to known antimicrobial peptides (AMPs) enables protease-activated release of caged AMPs.

FIG. 2A: Protease maturation often requires cleavage of an inhibitory propeptide domain. FIG. 2B: Design of propeptide-based fluorescent biosensors exclusively activated in the presence of pathogen-secreted proteases.

FIGS. 3A-3C illustrate that the IvaP propeptide I9 is both an inhibitor and substrate of IvaP. FIG. 3A: Purified IvaP was incubated with a fluorescent activity-based probe for serine hydrolases (FP-TAMRA) in the presence or absence of I9 for 5-60 min at room temperature prior to analysis by SDS-PAGE and fluorescence scanning. I9, I9 alone treated with probe for 60 min. PMSF, IvaP alone incubated with PMSF inhibitor prior to probe treatment for 60 min. FIG. 3B: Purified IvaP was incubated with I9 for 5-60 min at 37° C. prior to SDS-PAGE analysis and Coomassie Blue staining. IvaP, IvaP alone incubated at 37° C. for 60 min. I9, I9 alone incubated at 37° C. for 60 min. TCA, acid-treated IvaP incubated with I9 for 60 min. FIG. 3C: Concentrated supernatants from cultures of V. cholerae (Vc), Escherichia coli (Ec), and Salmonella enterica Typhimurium (Se) were incubated with FP-TAMRA in the presence or absence of I9 for 10 min at room temperature prior to gel-based analysis of serine hydrolase activity (top), followed by Coomassie Blue staining (bottom). Media, media alone incubated with probe in the presence or absence of I9.

FIGS. 4A-4D illustrate structure-guided mutation and characterization of I9 mutants for bioconjugation with environment-sensitive dyes. FIG. 4A: Homology model of the IvaP (blue) and I9 (green) complex generated by the Phyre2 server. IvaP active site appears in red. Dashed box highlights I9 residues along the predicted hydrophobic interface, including Y121 (arrow). FIG. 4B: Circular dichroism analysis of purified wild-type I9 (WT) and the representative I9 mutant Y121C indicating an α-β type fold. FIG. 4C: Purified IvaP was incubated with FP-TAMRA in the presence of WT I9 or Y121C I9 for 15-60 min at room temperature prior to gel-based analysis of serine hydrolase activity. FIG. 4D Representative thiol-reactive solvatochromic dyes.

FIG. 4E: Illustration of inhibition of the activity of IvaP (catalytic serine=5361) using the fluorescent serine hydrolase probe FP-TAMRA.

FIG. 4F: A non-limiting construct contemplated in the disclosure.

FIG. 5A: Wild type I9 does not contain native cysteines; thus, site-directed incorporation of cysteine residues into the I9 domain should yield variants with a functional handle for solvatochromic dye incorporation. The residues chosen for initial mutagenesis are highlighted. FIG. 5B: Solvatochromic dyes IANBD, IAEDANS, and BADMN are weakly fluorescent in aqueous solution but strongly fluorescent in hydrophobic environments such as protein-protein interfaces. MDCC is a conformation-sensitive dye.

FIG. 6A: Labeling efficiencies of four I9 Cys variants with IANBD, DMN, and IAEDANS. Bioconjugation reactions proceeded for 2 h at 25° C. for all variant dye combinations except S135C for which bioconjugation reactions were conducted overnight at 4° C. FIG. 6B: Increasing hydrophobicity (2.5-50% t-butanol) activates the fluorescence of Y121C I9-NBD.

FIG. 7A: Relative fluorescence change of I9-dye conjugates (3 μM) in the presence (F_{bound}) and absence (F_{unbound}) of IvaP (1 μM). The maximum fluorescence change (2-fold) was observed for the I128C I9-NBD conjugate in the presence of IvaP. These promising initial results suggest that the biosensor design could be further optimized by positioning the dyes in more hydrophobic, solvent-excluded pockets. FIG. 7B: Similar to wild type I9, Y121C I9-NBD inhibits FP-TAMRA labeling of IvaP, suggesting that bioconjugation does not affect binding.

FIG. 8 illustrates cleavage of an I9-AMP fusion (Pro-AMP, arrow) by IvaP. Pro-AMP was incubated with active IvaP or heat-killed (HK) IvaP for 30 min at room temperature prior to SDS-PAGE analysis and Coomassie Blue staining. Mass spectrometry analysis of the cleavage products was consistent with I9 cleavage.

FIG. 9A: Bioconjugation efficiencies were calculated by dividing the absorbance of each I9-dye pair by the extinction coefficient of the dye to give the dye concentration, which was subsequently divided by the concentration of the protein construct. FIG. 9B: I9 I128C-IANBD (3 μM) was incubated with or without purified IvaP (1 μM; preincubated with 1 μM PMSF for 15 min) at room temperature. Fluorescence measurements were taken immediately after probe addition in a multi-well plate reader. Curves represent the average of 3 technical replicates.

FIGS. 10A-10C depict that I9 E123C-MDCC can selectively detect V. cholerae in vitro. FIG. 10A: I9 E123C-MDCC (3 μM) was incubated with purified IvaP (1 μM preincubated with or without 1 μM PMSF for 15 min) for 60 min at room temperature. FIG. 10B: I9 E123C-MDCC (50 μM) was incubated with biofilm cultures of V. cholerae (Vc), V. parahaemolyticus (Vp), E. coli (Ec), or S. enterica Typhimurium (Se) at room temperature.

FIG. 10C: I9 E123C-MDCC (50 μM) was incubated with biofilm cultures of Vc or Vp or a mixed culture containing both species at room temperature. Fluorescence intensity was measured using a multiwell plate reader at the end of each experiment (FIG. 10A) or as a function of time (FIGS. 10B-10C). Curves in (FIG. b0A) and (FIG. 10C) are representative of data from 1 experiment; curves in (FIG. 10B) are the average of 3 biological replicates.

FIG. 11 depicts that MDCC is a conformation-sensitive dye.

FIG. 12 depicts that I9-DCC is selectively and reproducibly degraded by V. cholerae biofilm cultures as determined by both fluorescence spectroscopy (left) and gel-based (right) assays.

### DETAILED DESCRIPTION

The rapid and accurate diagnosis of infectious diseases is a major unmet clinical need, especially in resource-limited settings. Current diagnostic methods can be time-consuming or require specialized laboratory equipment, which limits their usefulness outside of a hospital. The identification of gastrointestinal pathogens is further complicated by the expansive gut microbiome, thus requiring diagnostic tools with high specificity for bacterial pathogens over commensals. It is therefore important to identify biomarkers of pathogenic bacterial infections and to design biosensors that detect the biomarker of infection with high specificity and sensitivity. Consequently, there is a need for technologies that can rapidly detect and/or selectively kill pathogenic bacteria within complex microbial communities.

The present disclosure relates, in one aspect, to the unexpected discovery that the native enzymatic machinery of pathogenic bacteria can be harnessed to develop biosensors and antimicrobials that target disease-causing microbes present in the body, such as but not limited to, in the digestive tract, respiratory tract, urinary tract, skin, and so forth. The present approach relates in one aspect to the use of proteases for the design of these precision tools.

Bacterial proteases, which are peptide-cleaving enzymes, are attractive biomarkers of infectious diseases as many pathogens require proteases to establish and maintain infections. Further, many proteases are produced in an inactive propeptide-bound form in which the propeptide domain binds to and inhibits the protease. Proteases are typically activated by cleavage of the propeptide domain during enzyme maturation. Propeptide domains bind to the active sites of their cognate proteases with high specificity, suggesting the propeptide-protease interaction may be useful as a biomarker-biosensor pair. For example, the serine protease IvaP is secreted by the cholera pathogen Vibrio cholerae during active infection. IvaP binds to, and is temporarily inhibited by, purified I9 in trans, suggesting that the I9 peptide can be engineered into a biosensor for IvaP. As demonstrated herein, one can exploit the I9-IvaP interaction to develop a prototype protease-dependent biosensor of V. cholerae infection.

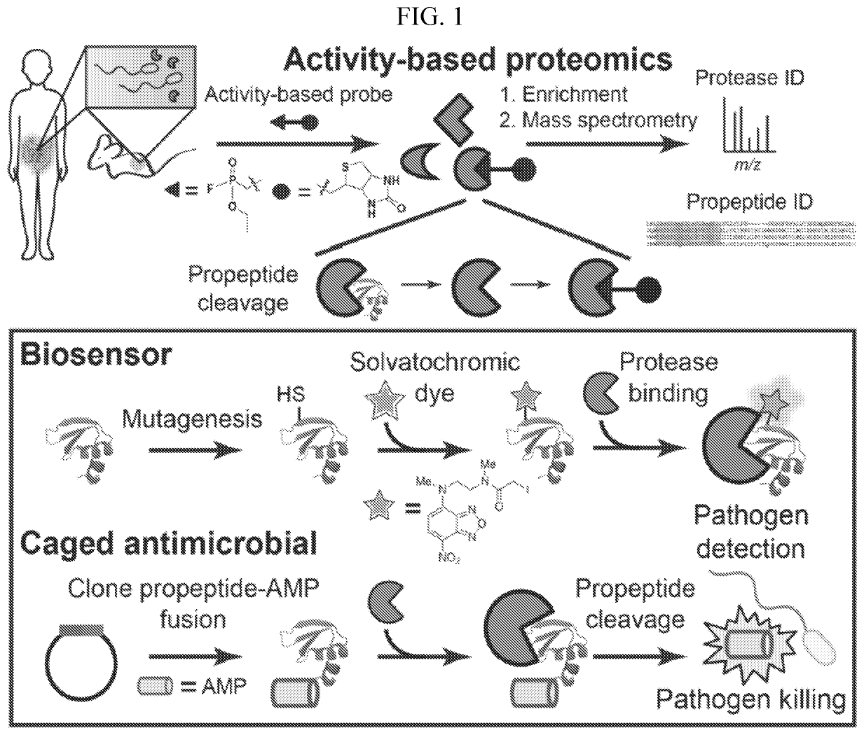

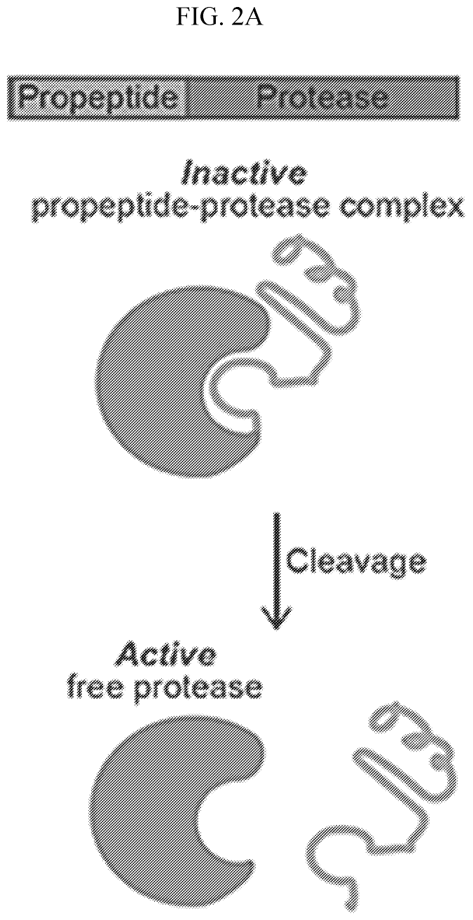

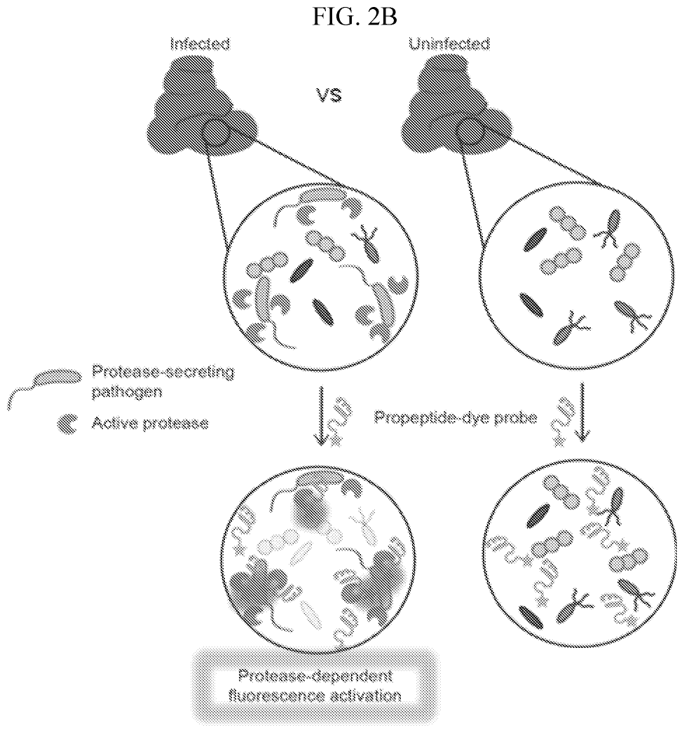

As demonstrated herein, propeptides can be adapted for the selective detection of protease-producing pathogens and controlled activation of caged antimicrobials (FIG. 1). In a non-limiting example, the propeptide of a bacterial protease that is active in human cholera can be used to engineer a prototype biosensor and antimicrobial system. In another non-limiting example, Clostridioides difficile (aka C. diff), which is a major cause of life-threatening intestinal inflammation commonly triggered by exposure to broad-spectrum antibiotics, can also be targeted. In another non-limiting example, P. aeruginosa, a bacterial pathogen associated with multi-drug resistance and high mortality rates in hospitalized patients and individuals with Cystic Fibrosis, can also be targeted.

In certain embodiments, the present technology can be applied from fundamental studies of microbial ecology to the diagnostic and therapeutic applications highlighted elsewhere herein.

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” “About” as used herein when referring to a measurable value such as an amount, a temporal duration, and the like, is meant to encompass variations of 20% or ±10%, in certain embodiments ±5%, in certain embodiments ±1%, in certain embodiments ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein the terms “alteration,” “defect,” “variation” or “mutation” refer to a mutation in a gene in a cell that affects the function, activity, expression (transcription or translation) or conformation of the polypeptide it encodes, including missense and nonsense mutations, insertions, deletions, frameshifts and premature terminations.

As used herein, the terms “conservative variation” or “conservative substitution” as used herein refers to the replacement of an amino acid residue by another, biologically similar residue. Conservative variations or substitutions are not likely to change the shape of the peptide chain. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine.

As used herein, a “construct” of the disclosure refers to a polypeptide (or polypeptide-containing molecule) comprising a propeptide, or a fragment or site directed mutant thereof.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “fragment,” as applied to a nucleic acid, refers to a subsequence of a larger nucleic acid. A “fragment” of a nucleic acid can be at least about 15, 50-100, 100-500, 500-1000, 1000-1500 nucleotides, 1500-2500, or 2500 nucleotides (and any integer value in between). As used herein, the term “fragment,” as applied to a protein or peptide, refers to a subsequence of a larger protein or peptide, and can be at least about 20, 50, 100, 200, 300 or 400 amino acids in length (and any integer value in between).

The term “functional equivalent” or “functional derivative” denotes, in the context of a functional derivative of an amino acid sequence, a molecule that retains a biological activity (either function or structural) that is substantially similar to that of sequences of any constructs shown herein. A functional derivative or equivalent may be a natural derivative or is prepared synthetically. The functionally-equivalent polypeptides of the disclosure can also be polypeptides identified using one or more techniques of structural and or sequence alignment known in art.

Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like. Typically, greater than 30% identity between two polypeptides is considered to be an indication of functional equivalence. Preferably, functionally equivalent polypeptides of the disclosure have a degree of sequence identity with the disclosed constructs of greater than 80%. More preferred polypeptides have degrees of identity of greater than 85%, 90%, 95%, 98% or 99%, respectively.

An “inducible” promoter is a nucleotide sequence that, when operably linked with a polynucleotide that encodes or specifies a gene product, causes the gene product to be produced in a cell substantially only when an inducer that corresponds to the promoter is present in the cell.

“Instructional material,” as that term is used herein, includes a publication, a recording, a diagram, or any other medium of expression that can be used to communicate the usefulness of the nucleic acid, peptide, and/or compound of the disclosure in the kit for identifying or alleviating or treating the various diseases or disorders recited herein.

“Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences that are normally adjacent to the fragment, i.e., the sequences adjacent to the fragment in a genome in which it naturally occurs. The term also applies to nucleic acids that have been substantially purified from other components which naturally accompany the nucleic acid, i.e., RNA or DNA or proteins, which naturally accompany it in the cell. The term therefore includes, for example, a recombinant DNA that is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA that is part of a hybrid gene encoding additional polypeptide sequence.

An “oligonucleotide” or “polynucleotide” is a nucleic acid ranging from at least 2, in certain embodiments at least 8, 15 or 25 nucleotides in length, but may be up to 50, 100, 1000, or 5000 nucleotides long or a compound that specifically hybridizes to a polynucleotide.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, the term “patient,” “individual” or “subject” refers to a human.

As used herein, the term “pharmaceutical composition” or “composition” refers to a mixture of at least one compound useful within the disclosure with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, the term “polypeptide” refers to a polymer composed of amino acid residues, related naturally occurring structural variants, and synthetic non-naturally occurring analogues thereof linked via peptide bonds.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements that are required for expression of the gene product. The promoter/regulatory sequence may for example be one that expresses the gene product in a tissue specific manner.

The term “recombinant polypeptide” as used herein is defined as a polypeptide produced by using recombinant DNA methods.

The term “recombinant DNA” as used herein is defined as DNA produced by joining pieces of DNA from different sources.

“Sample” or “biological sample” as used herein means a biological material isolated from a subject. The biological sample may contain any biological material suitable for detecting a mRNA, polypeptide or other marker of a physiologic or pathologic process in a subject, and may comprise fluid, tissue, cellular and/or non-cellular material obtained from the individual.

As used herein, “substantially purified” refers to being essentially free of other components. For example, a substantially purified polypeptide is a polypeptide that has been separated from other components with which it is normally associated in its naturally occurring state. Non-limiting embodiments include 95% purity, 99% purity, 99.5% purity, 99.9% purity and 100% purity.

The phrase “under transcriptional control” or “operatively linked” as used herein means that the promoter is in the correct location and orientation in relation to a polynucleotide to control the initiation of transcription by RNA polymerase and expression of the polynucleotide.

The term “transfected” or “transformed” or “transduced” as used herein refers to a process by which exogenous nucleic acid is transferred or introduced into the host cell. A “transfected” or “transformed” or “transduced” cell has been transfected, transformed or transduced with exogenous nucleic acid. The cell includes the primary subject cell and its progeny.

As used herein, the term “treatment” or “treating” is defined as the application or administration of a therapeutic agent, i.e., a compound useful within the disclosure (alone or in combination with another pharmaceutical agent), to a patient, or application or administration of a therapeutic agent to an isolated tissue or cell line from a patient (e.g., for diagnosis or ex vivo applications), who has a disease or disorder and/or a symptom of a disease or disorder, with the purpose to cure, heal, alleviate, relieve, alter, remedy, ameliorate, improve or affect the disease or disorder and/or the symptoms of the disease or disorder. Such treatments may be specifically tailored or modified, based on knowledge obtained from the field of pharmacogenomics.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide may differ in amino acid sequence by one or more substitutions, additions, or deletions in any combination. A variant of a nucleic acid or peptide may be a naturally occurring such as an allelic variant, or may be a variant that is not known to occur naturally. Non-naturally occurring variants of nucleic acids and peptides may be made by mutagenesis techniques or by direct synthesis.

A “vector” is a composition of matter that comprises an isolated nucleic acid and that may be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

As used herein, the term “virus” is defined as a particle consisting of nucleic acid (RNA or DNA) enclosed in a protein coat, with or without an outer lipid envelope, which is capable of transfecting the cell with its nucleic acid.

As used herein, the term “wild-type” refers to a gene or gene product isolated from a naturally occurring source. A wild-type gene is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. Naturally occurring mutants can be isolated; these are identified by the fact that they have altered characteristics (including altered nucleic acid sequences) when compared to the wild-type gene or gene product. As used herein, the amino acid sequence of IvaP from Vibrio cholerae, the alkaline serine protease corresponding to the gene VC 0157, is as follows:

```
SEQ ID NO: 1
                10 20 30 40
MFKKFLSLCI VSTFSVAATS ALAQPNQLVG KSSPQQLAPL
                50 60 70 80
MKAASGKGIK NQYIVVLKQP TTIMSNDLQA FQQFTQRSVN
                90 100 110 120
ALANKHALEI KNVFDSALSG FSAELTAEQL QALRADPNVD
              130 140 150 160
YIEQNQIITV NPIISASANA AQDNVTWGID RIDQRDLPLN
              170 180 190 200
RSYNYNYDGS GVTAYVIDTG IAFNHPEFGG RAKSGYDFID
              210 220 230 240
NDNDASDCQG HGTHVAGTIG GAQYGVAKNV NLVGVRVLGC
              250 260 270 280
DGSGSTEAIA RGIDWVAQNA SGPSVANLSL GGGISQAMDQ
              290 300 310 320
AVARLVQRGV TAVIAAGNDN KDACQVSPAR EPSGITVGST
              330 340 350 360
TNNDGRSNFS NWGNCVQIFA PGSDVTSASH KGGTTTMSGT
              370 380 390 400
SMASPHVAGV AALYLQENKN LSPNQIKTLL SDRSTKGKVS
              410 420 430 440
DTQGTPNKLL YSLTDNNTTP NPEPNPQPEP QPQPDSQLTN
              450 460 470 480
GKVVTGISGK QGELKKFYID VPAGRRLSIE TNGGTGNLDL
              490 500 510 520
YVRLGIEPEP FAWDCASYRN GNNEVCTFPN TREGRHFITL
              530
YGTTEFNNVS LVARY
```

In certain embodiments, in SEQ ID NO:1, the signal peptide comprises amino acid residues 1-23, or a biologically equivalent fragment and/or mutant thereof, and the chain comprises amino acid residues 24-535, or a biologically equivalent fragment and/or mutant thereof. In certain embodiments, the I9 inhibitor domain comprises amino acid residues 24-134, 52-101, and/or 52-128 of SEQ ID NO:1. The S8 peptidase domain is defined by amino acid residues 169-395 of SEQ ID NO:1.

As used herein, the amino acid sequence of the C. difficile protease CspB is as follows:

```
SEQ ID NO: 2:
                10 20 30 40
MIIINYELIV KYNGDILRLE EELGVSVEIL NSSYAIITSS
                50 60 70 80
NEEDVNILLT YPEIEFIEKP FILQTQDVQS FSSTGITGFK
                90 100 110 120
NRTGLTGKGT IIGIIDSGID YTLPVERDSD GRSKILYYWD
              130 140 150 160
QSIQGNPPEG FREGTLYTNE DINNAIDGSM YIPISTTSLH
              170 180 190 200
GTHVAGICAT IASDARIIVV RVGNIQTDIF SRSTEFMRAI
              210 220 230 240
KFILDRALEL PMPVTLNISY GSNEGSHRGT SLFEQYIDDM
              250 260 270 280
CLFWKNNIVV AAGNNADKGG HKRIRLQNNI TEEVEFIVGE
              290 300 310 320
GERILNINIW PDFVDDFSVH LVNPSNNQTQ AISLTSGEIP
              330 340 350 360
NTLGETPITG YFYPIAPYSL TRRVTLQLSS NTQITPGLWK
              370 380 390 400
IVFEPIDIVT GNVNIYLPTS EGLNRNTRFL IPTQELTVTV
              410 420 430 440
PGTASRVITV GSFNSRTDIV SIFSGEGDTQ LGVFKPDLLA
              450 460 470 480
PGEDIVSFLP GGTSGALTGT SMATPHVTGV CSLFMEWGIV
              490 500 510 520
NGNDLFLYSQ KLRALLLKGA RRLSNQSYPN NSSGFGFLNL
              530 540
SDIDLYTLSN INQDLETEDM GYRSINKS
```

In certain embodiments, in SEQ ID NO:2, the chain comprises amino acid residues 1-548, or biologically equivalent fragment and/or mutant thereof. In certain embodiments, the I9 inhibitor domain comprises amino acid residues 1-66 of SEQ ID NO:2. The peptidase domain is defined by amino acid residues 67-548 of SEQ ID NO:2.

As used herein, the amino acid sequence of the P. aeruginosa Protease IV is as follows:

```
SEQ ID NO: 3:
                10 20 30 40
MHKRTYLNAC LVLALAAGAS QALAAPGASE MAGDVAVLQA
                50 60 70 80
SPASTGHARF ANPNAAISAA GIHFAAPPAR RVARAAPLAP
                90 100 110 120
KPGTPLQVGV GLKTATPEID LATLEWIDTP DGRHTARFPI
              130 140 150 160
SAAGAASLRA AIRLETRSGS LPDDVLLHFA GDGKEIFEAS
              170 180 190 200
GKDLSVNRPY WVPVIEGDTL TVELVLPANL QPGDLRLSVP
              210 220 230 240
QVSYFADSLY KAGYRDGFGA SGSCEVDAVC ATQSGTRAYD
              250 260 270 280
NATAAVAKMV FTNSADGGSY ICTGTLLNNG NSPKRQLFWS
              290 300 310 320
AAHCIEDQAT AATLQTIWFY NTTQCYGDAS TINQEVTVLT
              330 340 350 360
GGANILHRDA KRDTLLLELK RTPPAGVFYQ GWSATPIANG
              370 380 390 400
SLGHDIHHPR GDAKKYSQGN VSAVGVTYDG HTALTRVDWP
              410 420 430 440
SAVVEGGSSG SGLLTVAGDG SYQLRGGLYG GPSYCGAPTS
              450 460
QRNDYFSDFE GVYSQISRYF AP
```

In certain embodiments, in SEQ ID NO:3, the signal peptide comprises amino acid residues 1-24, or a biologically equivalent fragment and/or mutant thereof, and the chain comprises amino acid residues 25-462, or a biologically equivalent fragment and/or mutant thereof. In certain embodiments, the propeptide inhibitor domain comprises amino acid residues 25-211 of SEQ ID NO:3. The peptidase domain is defined by amino acid residues 212-462 of SEQ ID NO:3.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Polypeptides/Proteins

In one aspect, the disclosure contemplates any propeptide relating to a pathogenic bacterium protease, as well as any mutants, fragments, and/conjugates of such propeptides. Various non-limiting embodiments of the disclosure are described herein, and each can be combined with any other embodiment(s) in any way useful within the disclosure.

Propeptides are proteinase propeptide inhibitors (sometimes referred to as activation peptides) responsible for the modulation of folding and activity of the peptidase pro-enzyme or zymogen. The prosegment docks into the enzyme, shielding the substrate binding site, thereby promoting inhibition of the enzyme. Several such propeptides share a similar topology, despite often low sequence identities. The propeptide region has an open-sandwich antiparallel-alpha/antiparallel-beta fold, with two alpha-helices and four beta-strands with a (beta/alpha/beta)×2 topology. The peptidase inhibitor I9 family contains the propeptide domain at the N-terminus of peptidases belonging to MEROPS family S8A, subtilisins. The propeptide is removed by proteolytic cleavage, thus activating the enzyme. In certain embodiments, the protease is a subtilisin, as known in the art. In certain embodiments, the propeptide is a I9 propeptide, as known in the art. In certain embodiments, the propeptide is a full length I9 propeptide.

In certain embodiments, the propeptide comprises the propeptide I9, or a mutant, fragment, and/or conjugate thereof, for IvaP protease from Vibrio cholerae. In certain embodiments, the propeptide I9 corresponds to amino acid residues 52-128 of SEQ ID NO:1.

In certain embodiments, the propeptide comprises the propeptide I9, or a mutant, fragment, and/or conjugate thereof, for CspB protease of C. difficile. In certain embodiments, the propeptide I9 corresponds to amino acid residues 1-66 of SEQ ID NO:2.

In certain embodiments, the propeptide comprises the propeptide inhibitor domain, or a mutant, fragment, and/or conjugate thereof, for Protease IV of P. aeruginosa. In certain embodiments, the propeptide inhibitor domain corresponds to amino acid residues 25-211 of SEQ ID NO:3.

In one aspect, the disclosure contemplates derivatives of any propeptide useful within the disclosure. In certain embodiments, these derivatives include conjugates of the propeptide with a detectable label, such as a fluorophore, chromophore, or any other detectable label that allows for the detection of the interaction of the derivatized propeptide with the protease.

In certain embodiments, the fluorophore is a solvatochromic dye. In certain embodiments, the dye is weakly fluorescent in aqueous solution but strongly fluorescent in hydrophobic environments such as protein-protein interfaces of a pathogenic bacterium. Non-limiting examples of these dyes include:

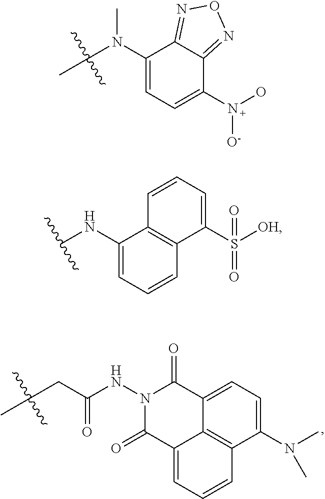 [SMILES: *N(C)c1ccc(N([O-])O)c2nonc12 ; *Nc1cccc2c(S(=O)(=O)O)cccc12 ; *CC(=O)NN1C(=O)c2cccc3c(N(C)C)ccc(c23)C1=O]

charge transfer dyes (a class of solvatochromic dyes), such as but not limited to: PRODAN, Dansyl, anthradan, FR0, PA, 4DMP, 6DMN, Fluoroprobe, Dapoxyl, Nile red, DCDHF-6, 3MC-2 (Acc. Chem. Res. 2017, 50(2):366-375), Pyrene and its analogs, and coumarin and its analogs;

excited-state intramolecular proton transfer dyes (a class of solvatochromic dyes), such as but not limited to: 31H, 3HC, DMA-3H1F, FA, 3HQ, HBON, diCN-HBO, SAN (Acc. Chem. Res. 2017, 50(2):366-375);

merocyanine dyes (a class of solvatochromic dyes), such as but not limited to: Bioconjug Chem. 2013, 24(2):215-223. doi:10.1021/bc3005073.

In certain embodiments, the fluorophore is a conformation-sensitive fluorophore with a fluorescence intensity determined by the conformational state of the molecule. In certain embodiments, the conformation-sensitive fluorophore is DCC and its derivatives:

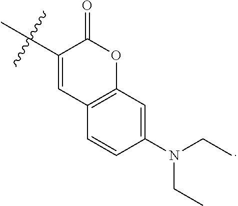 [SMILES: *c1cc2ccc(N(CC)CC)cc2oc1=O]

In certain embodiments, constrained rotation enhances DCC fluorescence, whereas an increase in rotational flexibility decreases dye fluorescence. Therefore, in one embodiment wherein the DCC fluorophore is conjugated with a propeptide, the DDC fluorophore emits a weaker detectable signal once the propeptide is cleaved.

In certain embodiments, the fluorophore is a TCC probe. In certain embodiments, the probe is a turn-on fluorescent dye that is activated by a conjugate addition and cyclization reaction with a nucleophilic amino acid such as on the protease surface. Non-limiting examples of these dyes include TCC-1 and its derivatives (Cell Chem Biol. 2020, 27(3):334-349. ell. doi:10.1016/j.chembiol.2020.01.006):

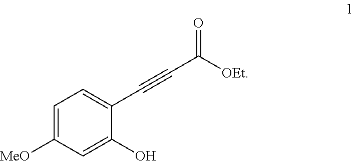 [SMILES: CCOC(=O)C#Cc1ccc(OC)cc1O]

In certain embodiments, these derivatives include conjugates of the propeptide with a detectable label, such as a fluorophore, chromophore, or any other detectable label, wherein the labels emits a stronger detectable signal once the propeptide is cleaved. Non-limiting examples of these dyes include:

fluorophores with FRET-based quenchers that are uncoupled by propeptide cleavage: DABCYL (4-(dimethylaminoazo)benzene-4-carboxylic acid) quencher and its derivatives, 5-FAM (5-Carboxyfluorescein) and its derivatives, fluorescein isothiocyanate and its derivatives (see EBioMedicine. 2018; 38:248-256. doi:10.1016/j.ebiom.2018.11.031 and Anal Chem. 2011; 83(7):2511-2517. doi:10.1021/ac102764v);

cleavage-activated chromophores, such as but not limited top-nitroaniline and its derivatives.

The detectable labels contemplated within the disclosure can be conjugated to the propeptide using methods known in the art. The detectable labels can be conjugated to the propeptide through a direct bond or through a linker. Reactions used for such attachment are known to those skilled in the art, such for example amide bond formation (wherein the detectable label or linker-derivatized detectable label has a free primary or secondary amine group and the propeptide has a free carboxylic acid group, or the detectable label or linker-derivatized detectable label has a free carboxylic acid group and the propeptide has a free primary or secondary amine group), nucleophilic displacement (wherein the detectable label or linker-derivatized detectable label has a nucleophilic group and the propeptide has a leaving group capable of being displaced by the nucleophile, or the propeptide has a nucleophilic group and the detectable label or linker-derivatized detectable label has a leaving group capable of being displaced by the nucleophile), and so forth.

In certain embodiments, the detectable label is attached to a residue of the propeptide so that the label is present in the interface of the propeptide-protease complex once the propeptide binds to the protease. In this way, the physical environment of the label changes once the propeptide-protease complex is formed, and experiences some physical or physico-chemical change that allows for detecting formation of the propeptide-protease complex. For example, if the label is a solvatochromic dye, formation of the propeptide-protease complex causes an enhancement of the dye fluorescence, as the dye migrates from an aqueous environment to a hydrophobic environment.

In certain embodiments, the label or the linker-containing label is conjugated to a naturally occurring amino acid residue of the propeptide. Such residues include but are not limited to Asp, Glu, Lys, Ser, Thr, Cys, and others, wherein a covalent bond can be formed between the residue and the label/linker-containing label. In other embodiments, the linker-containing label comprises one or more natural and/or unnatural amino acids, or any other linker known in the art. In other embodiments, the label or linker-containing label is conjugated to a mutated residue of the propeptide. The mutation can be introduced in the propeptide to enable chemical coupling of the label or linker-containing label, but should not disturb the propeptide-protease interaction to a significant extent, so that the derivatized propeptide can still bind to the protease under physiological conditions. Examples of such mutations include, in a non-limiting embodiment, introduction of Cys residues, which are capable of displacing leaving groups (such as a halogen, mesylate, tosylate, and the like) in the label or linker-containing label. Identification of residues that can be mutated in the propeptide can be done, for example, by alanine scanning of the propeptide or examination of the propeptide-protease structure through crystallography, NMR, or any other appropriate physical method. In the case of I9 for IvaP from Vibrio cholerae, I9 residues that can be mutated to Cys for facilitating label derivatization include, but are not limited to, A35, A44, K42, 154, V79, F94, A97, S102, A112, Y121, E123, N125, Q126, 1128, Asn131, and/or Ser135, N144, and any double or multiple mutants thereof. In certain embodiments, the I9 propeptide is a full length I9 from Vibrio cholerae comprising a E123C mutation. In the case of I9 for CspB of C. difficile, I9 residues that can be mutated to Cys for facilitating label derivatization include, but are not limited to, 136, E58, F61, L63, Q64, T65, and any double or multiple mutants thereof. In the case of the propeptide inhibitor for Protease IV of P. aeruginosa, propeptide residues that can be mutated to Cys for facilitating label derivatization include, but are not limited to, K211.

In another aspect, the disclosure further contemplates conjugates of a propeptide useful within the disclosure, or a fragment, mutant, or conjugate thereof, with a biologically active agent, such as but not limited to an antimicrobial peptide (AMP). In certain embodiments, the propeptide is conjugated directly or through a linker to the N-terminus of the AMP. In such construct, the N-terminus-blocked AMP has no antimicrobial activity.

However, when the conjugate of the propeptide and the AMP binds to the propeptide-specific protease, the AMP or a biologically active derivative thereof is released to exert its antimicrobial activity in the vicinity of the protease. Non-limiting examples of AMPs contemplated within the disclosure include SMAP-29, SMAP-29B, SMAP-29D, LL-29, LL-37, CRAMP, eCATH-1, eCATH-2, PMAP-23, PMAP-36, RL-37, CAP-11, CAP-18, BMAP-27, BMAP-27C, BMAP-34, PaDBS1R6F10, G12.21, G14.15, and variants thereof.

In another aspect, the disclosure further contemplates conjugates of a propeptide useful within the disclosure, or a fragment, mutant, or conjugate thereof, with an electrophilic group, such as but not limited to a fluorophosphonate, diphenylphosphonate, acyloxymethylketone (which binds to Cys protease), sulfonyl fluoride, chloromethyl ketone, bromo/chloromethylketones (which binds to Cys) iodoacetamide (which binds to Cys), and maleimide (which binds to Cys), which is capable of covalently labeling and inactivating the protease once the propeptide binds to the protease. In certain embodiments, the propeptide is conjugated directly or through a linker to the electrophilic group, whereby the propeptide has a natural or mutated residue anywhere in its structure that can be coupled to the electrophilic group and/or to a linker comprising the electrophilic group. In certain embodiments, the C-terminus of the propeptide is conjugated directly or through a linker to the electrophilic group. In such construct, when the conjugate of the propeptide and the electrophilic group binds to the propeptide-specific protease, the electrophilic group is capable of reacting with the catalytic serine of the protease, inactivating the protease.

The linkers contemplated in any aspect of the disclosure can be any linker known in the art, as long as they do not significantly disturb or modify the biological activity of the components of the construct in which they are included. In certain embodiments, the linker comprises from 1 to about 100 amino acids. In certain embodiments, the linker comprises about 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, or 100 amino acids. In certain embodiments, the linker comprises from about 1 to about 50 residues selected from O, CH₂, CH₂CH₂, —C(═O)NH—, —C(═O)NCH₃—, CH(CH₃)CH₂, CH₂CH₂O, C(CH₃)CH₂O, and

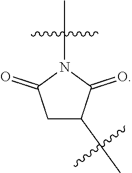 [SMILES: *C1CC(=O)N(*)C1=O]

In certain embodiments, the linker comprises a polyethylene glycol and/or an oligomeric ethylene glycol. In certain embodiments, the linker is self-immolative (see, for example, Tranoy-Opalinski, et al., 2008, Anticancer Agents Med. Chem. 8(6):618-637; Blencowe, et al., 2011, Polym. Chem. 2:773-790). In certain embodiments, the linker is

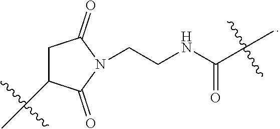 [SMILES: *C(=O)NCCN1C(=O)CC(*)C1=O]

In some embodiments, the combination of the fluorophore with the linker has the following structure:

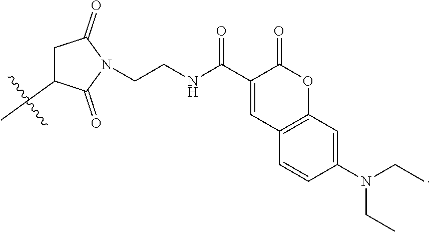 [SMILES: *C1CC(=O)N(CCNC(=O)c2cc3ccc(N(CC)CC)cc3oc2=O)C1=O]

In certain embodiments, the combination of the fluorophore and the linker is formed from the reaction of the maleimide of MDCC with a thiol on the propeptide. In certain embodiments, MDCC has the following structure:

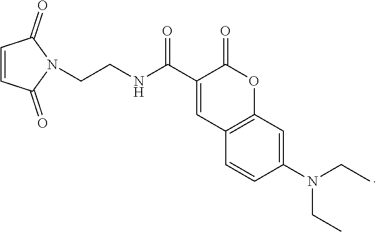 [SMILES: CCN(CC)c1ccc2cc(C(=O)NCCN3C(=O)C=CC3=O)c(=O)oc2c1]

Although not wishing to be limited by theory, it is believed that the cleavage of a propeptide comprising MDCC by IvaP enhances the fluorescence of DCC (as MDCC), most likely by cleaving the peptide backbone into smaller fragments that can diffuse and rotate more freely in solution.

### Vectors and Cells

Also provided herein are nucleic acids that encode any one of the constructs of the disclosure. The disclosure further provides vectors, such as expression vectors, that comprise such nucleic acids. Also provided are a cell, cells, or a plurality of cells (e.g., mammalian cells) that comprise any one of the nucleic acids, vectors, or expression vectors described herein. Also provided are methods for producing a protein (e.g., any one of constructs of the disclosure), the methods in certain embodiments comprising culturing the cell, cells, or plurality of cells under conditions suitable for expression of the protein by the cell or cells from the nucleic acid, vector, or expression vector. The methods can also include purifying the protein from the cell, cells, or plurality of cells, or from the media in which the cell, cells, or plurality of cells were cultured. In addition, the disclosure provides proteins purified by any such methods.

The disclosure further provides an autonomously replicating or an integrative mammalian cell vector comprising a recombinant nucleic acid encoding a construct of the disclosure. In certain embodiments, the vector comprises a plasmid or a virus. In other embodiments, the vector comprises a mammalian cell expression vector. In yet other embodiments, the vector further comprises at least one nucleic acid sequence that directs and/or controls expression of the construct.

In yet another aspect, the disclosure provides an isolated host cell comprising a vector of the disclosure. In certain embodiments, the cell is a non-human cell. In other embodiments, the cell is mammalian. In yet other embodiments, the vector of the disclosure comprises a recombinant nucleic acid encoding a given construct comprising a construct of the disclosure and a signal peptide. In yet other embodiments, the given construct is proteolytically processed upon secretion from a cell to yield the construct of the disclosure.

### Production and Purification of Polypeptides/Proteins

In certain embodiments, the polypeptides and/or proteins of the disclosure are produced using recombinant methods. In certain embodiments, the polypeptides and/or proteins of the disclosure are produced using native chemical ligation. In certain embodiments, the polypeptides and/or proteins of the disclosure are produced using solid-phase protein chemistry. In certain embodiments, the polypeptides and/or proteins of the disclosure are produced using any combination of any of the methods described herein.

Many expression systems are known can be used for the production of proteins, including bacteria (for example E. coli and Bacillus subtilis), yeasts (for example Saccharomyces cerevisiae, Kluyveronmyces lactis and Pichia pastoris), filamentous fungi (for example Aspergillus), plant cells, animal cells and insect cells. The desired protein can be produced in conventional ways, for example from a coding sequence inserted in the host chromosome or on a free plasmid.

The yeasts can be transformed with a coding sequence for the desired protein in any of the usual ways, for example electroporation. Methods for transformation of yeast by electroporation are disclosed in Becker & Guarente, 1990, Methods Enzymol. 194: 182.

Successfully transformed cells, i.e., cells that contain a DNA construct of the present disclosure, can be identified by well-known techniques. For example, cells resulting from the introduction of an expression construct can be grown to produce the desired polypeptide.

Cells can be harvested and lysed and their DNA content examined for the presence of the DNA using a method, such as that described by Southern, 1975, J. Mol. Biol, 98:503 and/or Berent, et al., 1985, Biotech 3:208. Alternatively, the presence of the protein in the supernatant can be detected using antibodies.

Useful yeast plasmid vectors include pRS403-406 and pRS413-416 and are generally available fron1 Strat:1.gene Cloning Systems, La Jolla, Calif., USA Plasmids pRS403, pRS404, pRS405 and pRS406 are Yeast Integrating plasmids (Y1ps) and incorporate the yeast selectable markers I-11S3, TRP1, LEU2 and 1JRA3. Plasmids pRS413 416 are Yeast Centromere plasmids (YCps).

A variety of methods have been developed to operably link DNA to vectors via complementary cohesive termini. For instance, complementary homopolymer tract can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion, is treated with bacteriophage T4 DNA polymerase or E. coli DNA polymerase I, which are enzymes that remove protruding, 3′-single-stranded termini with their 3′-5′-exonucleolytic activities, and fill in recessed 3′-ends with their polymerizing activities.

The combination of these activities thus generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Clones of single, stably transfected cells are then established and screened for high expressing clones of the desired fusion protein. Purification of protein can be accomplished using a combination of standard purification techniques known in the art.

### Methods

The disclosure includes a method of identifying a pathogenic bacterium in a sample, the method comprising contacting the sample with a construct comprising a pathogenic bacterium protease propeptide conjugated to a detectable label, wherein the bacterium produces the protease that binds to the propeptide, and monitoring a detectable signal for the label before and after the contacting. In certain embodiments, a qualitative and/or quantitative change in the detectable signal upon contacting indicates that the pathogenic bacterium is present in the sample. In certain embodiments, the sample comprises another bacterium that does not produce the protease that binds to the propeptide. In certain embodiments, the detectable label comprises a fluorophore, chromophore, magnetic nanoparticle, or any other detectable label known in the art and/or described elsewhere herein.

The disclosure includes a method of killing a pathogenic bacterium, or reducing or preventing growth of a pathogenic bacterium, in a sample, the method comprising contacting the bacterium with a construct comprising a pathogenic bacterium protease propeptide conjugated to an antimicrobial peptide (AMP) and/or a therapeutically active small molecule (such as, but not limited to, vancomycin, metronidazole, and so forth) and/or a detectable label that emits a stronger detectable signal once the propeptide is cleaved, wherein the bacterium produces the protease that binds to the propeptide. In certain embodiments, the AMP is released from the construct. In certain embodiments, the contacting is performed in vivo in a subject infected by the pathogenic bacterium. In certain embodiments, the pathogenic bacteria is in the gut of the subject. In certain embodiments, non-pathogenic bacteria in the gut of the subject are not significantly killed, or wherein growth of non-pathogenic bacteria in the gut is not significantly reduced or prevented.

The disclosure includes a method of inactivating a protease of a pathogenic bacterium, the method comprising contacting the bacterium with a construct comprising a pathogenic bacterium protease propeptide conjugated to an electrophilic agent, wherein the bacterium produces the protease that binds to the propeptide.

In certain embodiments, the construct is administered acutely or chronically to the subject. In other embodiments, the construct is administered locally, regionally, parenterally or systemically to the subject. In other embodiments, the construct is administered as an engineered probiotic.

In certain embodiments, the subject is a mammal. In other embodiments, the mammal is human.

In certain embodiments, the construct is administered by at least one route selected from the group consisting of subcutaneous, oral, aerosol, inhalational, rectal, vaginal, transdermal, subcutaneous, intranasal, buccal, sublingual, parenteral, intrathecal, intragastrical, ophthalmic, pulmonary, and topical. In other embodiments, the construct is administered to the subject as a pharmaceutical composition further comprising at least one pharmaceutically acceptable carrier.

It will be appreciated by one of skill in the art, when armed with the present disclosure including the methods detailed herein, that the disclosure is not limited to treatment of a disease or disorder once it is established. Particularly, the symptoms of the disease or disorder need not have manifested to the point of detriment to the subject; indeed, the disease or disorder need not be detected in a subject before treatment is administered.

That is, significant pathology from disease or disorder does not have to occur before the present disclosure may provide benefit.

Thus, the present disclosure, as described more fully herein, includes a method for preventing diseases and disorders in a subject, in that a construct of the disclosure, as discussed elsewhere herein, can be administered to a subject prior to the onset of the disease or disorder, thereby preventing the disease or disorder from developing. Particularly, where the symptoms of the disease or disorder have not manifested to the point of detriment to the subject; indeed, the disease or disorder need not be detected in a subject before treatment is administered. That is, significant pathology from the disease or disorder does not have to occur before the present disclosure may provide benefit. Therefore, the present disclosure includes methods for preventing or delaying onset, or reducing progression or growth, of a disease or disorder in a subject, in that a construct of the disclosure can be administered to a subject prior to detection of the disease or disorder.

Armed with the disclosure herein, one skilled in the art would thus appreciate that the prevention of a disease or disorder in a subject encompasses administering to a subject a construct of the disclosure as a preventative measure against the disease or disorder.

### Pharmaceutical Compositions and Formulations

The disclosure provides pharmaceutical compositions comprising a construct of the disclosure within the methods described herein.

Such a pharmaceutical composition is in a form suitable for administration to a subject, or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

In certain embodiments, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 500 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between about 0.1% and about 100% (w/w) active ingredient.

Pharmaceutical compositions that are useful in the methods of the disclosure may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

### Administration/Dosing

The regimen of administration may affect what constitutes an effective amount. For example, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. Dosage is determined based on the biological activity of the therapeutic compound which in turn depends on the half-life and the area under the plasma time of the therapeutic compound curve.

For example, several divided doses may be administered daily or the dose may be proportionally reduced as indicated by the exigencies of the therapeutic situation. A non-limiting example of an effective dose range for a therapeutic compound of the disclosure is from about 0.01 and 50 mg/kg of body weight/per day. In certain embodiments, the effective dose range for a therapeutic compound of the disclosure is from about 50 ng to 500 ng/kg, preferably 100 ng to 300 ng/kg of bodyweight. One of ordinary skill in the art would be able to study the relevant factors and make the determination regarding the effective amount of the therapeutic compound without undue experimentation.

The compound can be administered to a patient as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the patient.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In certain embodiments, the compositions of the disclosure are administered to the patient in dosages that range from one to five times per day or more. In other embodiments, the compositions of the disclosure are administered to the patient in range of dosages that include, but are not limited to, once every day, every two, days, every three days to once a week, and once every two weeks. The frequency of administration of the various combination compositions of the disclosure varies from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physical taking all other factors about the patient into account.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

### Routes of Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. The formulations and compositions that would be useful in the present disclosure are not limited to the particular formulations and compositions that are described herein.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

### Additional Administration Forms

Additional dosage forms of this disclosure include dosage forms as described in U.S. Pat. Nos. 6,340,475, 6,488,962, 6,451,808, 5,972,389, 5,582,837, and 5,007,790. Additional dosage forms of this disclosure also include dosage forms as described in U.S. Patent Applications Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

Controlled- or sustained-release formulations of a pharmaceutical composition of the disclosure may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, ionic liquids, multilayer coatings, microparticles, nanoparticles, liposomes, or microspheres or a combination thereof to provide the desired release profile in varying proportions. Single unit dosage forms suitable for oral administration, such as tablets, capsules, gelcaps, and caplets, which are adapted for controlled-release are encompassed by the present disclosure. In one embodiment, a pharmaceutical composition of the disclosure comprises an ionic liquid. In one embodiment, the ionic liquid enhances ADMET, especially when the disclosed constructs are administered to a subject via a transdermal route. In one embodiment, the ionic liquid has antimicrobial activity. In one embodiment, an ionic liquid with antimicrobial activity works synergistically with any additional antimicrobials in the pharmaceutical composition, including but not limited to, an AMP in the disclosed construct.

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form. For sustained release, the compounds may be formulated with a suitable polymer or hydrophobic material that provides sustained release properties to the compounds. As such, the compounds for use the method of the disclosure may be administered in the form of microparticles, for example, by injection or in the form of wafers or discs by implantation. In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours. The term pulsatile release is used herein in its conventional sense to refer to a drug formulation that provides release of the drug in such a way as to produce pulsed plasma profiles of the drug after drug administration. The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction and preparation conditions, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### Examples

The disclosure is now described with reference to the following Examples. These Examples are provided for the purpose of illustration only, and the disclosure is not limited to these Examples, but rather encompasses all variations that are evident as a result of the teachings provided herein.

### Methods and Materials

Protein Overexpression and Purification. IvaP was purified as previously described (J Biol Chem. 2019; 294(25):9888-9900. doi:10.1074/jbc.RA119.007745). The I9 domain (residues 24-134) was overexpressed in E. coli BL21 (DE3) cells harboring I9-pET28b(+) with an N-terminal hexa-histidine tag. Saturated overnight cultures were diluted 1,000-fold into sterile LB supplemented with 50 μg/mL kanamycin. Cells were grown at 37° C. and 250 rpm to OD600=0.6, at which point overexpression was induced upon addition of 0.5 mM IPTG followed by a 5-hour incubation at 37° C. with shaking at 250 rpm. Cells were collected by centrifugation at 17,000×g and 4° C. for 10 min and then resuspended in 6 mL Ni Buffer A (20 mM Tris, pH 7.4, 500 mM NaCl, 30 mM imidazole, 1 mM TCEP) supplemented with 5 μL benzonase and 1 tablet of EDTA-free protease inhibitor tablets for His-tagged proteins (Sigma) per gram of cell pellet). Resuspended cells were lysed via microfluidizer (Microfluidics Corporation; 10,000 psi at 4° C., two times) and then the cell debris were removed by centrifugation at 21,000×g and 4° C. for 30 min. The supernatant was passed through a 0.22 μm filter and then loaded onto a 1 mL HisTrap FF column (GE Healthcare) preequilibrated with Ni Buffer A. The column was then washed with 10 column volumes of Ni buffer A and then I9 was eluted in a stepwise gradient of 0-100% Ni buffer B (20 mM Tris, pH 7.4, 500 mM NaCl, 500 mM imidazole, and 1 mM TCEP) over 20 column volumes. Fractions containing I9 (determined by SDS-PAGE gel electrophoresis) were combined and then concentrated to less than 500 μL. Particulates were removed from the protein solution by centrifugation at 21,000×g and 4° C. for 30 min. The supernatant was loaded onto a Superdex 200 Increase 10/300 GL column (GE Healthcare) and eluted over 1 column volume in gel filtration buffer (50 mM Tris, 150 mM NaCl, pH 7.4) at 4° C. I9 elutes as a single peak consistent with a monomer. Fractions containing I9 (determined by SDS-PAGE gel electrophoresis) were combined, and concentrated to at least 100 μM. The I9 concentration was determined via A280 of the denatured protein in 6 M guanidine-HCl. Protein was flash frozen in liquid nitrogen and stored at −80° C.

Protein Characterization. The binding of I9 to IvaP was determined using the FP-TAMRA competition assay as previously described (J Biol Chem. 2019; 294(25):9888-9900. doi:10.1074/jbc.RA119.007745). Secondary structure was determined using circular dichroism (Applied Photophysics). A solution of I9 (15 μM, 400 μL) in gel filtration buffer was added to a 1 mm quartz cuvette (Starna). A wavelength scan from 350 nm to 185 nm was collected with a 2 second averaging time and 1 nm step at room temperature.

Dye bioconjugation. I9 (at least 250 μL of 100 μM) was incubated with 1 mM TCEP overnight at 4° C. to ensure all cysteines were fully reduced. The solution was then treated with 100 mM of IANBD, 4-BA-DMN, IEDANS (prepared as a 50 mM stock in DMSO) or 250 μM of MDCC (prepared as a 50 mM stock in DMF) and the suspension was incubated at room temperature in the dark for at least 2 hours. Unreacted dye was removed by size exclusion using NAP-5 columns (Illustra) preequilibrated with 50 mM Tris, 150 mM NaCl, pH 7.4 at room temperature in the dark. Eluted protein was concentrated and then the concentration of protein was determined via A280. The concentration of the dye was determined by UV absorption (λₘₐₓ^{NBD}: 478 nm, ε=25,000 M⁻¹cm⁻¹; λₘₐₓ^{DMN}: 440 nm, ε=8,800 M⁻¹cm⁻¹; λₘₐₓ^{DANS}: 336 nm, ε=5,700 M⁻¹cm⁻¹, λₘₐₓ^{DCC}: 435 nm, ε=46,800 M⁻¹cm⁻¹) and the labeling efficiency was determined by dividing the calculated dye concentration by the calculated protein concentration. I9-dye conjugates were flash frozen in liquid nitrogen and stored at −80° C.

Fluorescence plate reader assays. A solution of I9-dye probe (3 μM) with purified IvaP (1 μM preincubated with or without 1 μM PMSF for 15 min) in 50 mM Tris, 150 mM NaCl, pH 7.4 (100 μL total) was added to a 96-well black-walled microplate. Emission wavelength scans were collected from 750 nm at a single excitation (NBD: 455 nm, DMN: 405 nm, DANS: 336 nm, DCC: 430 nm) at room temperature. For time-course analysis with whole-cell cultures, I9-DCC (50 μM) was added to biofilm cultures (100 μL total) using a multichannel pipette. Directly following probe addition, the fluorescence emission at 475 nm (excitation: 430 nm) was monitored over time at room temperature.

### Example 1

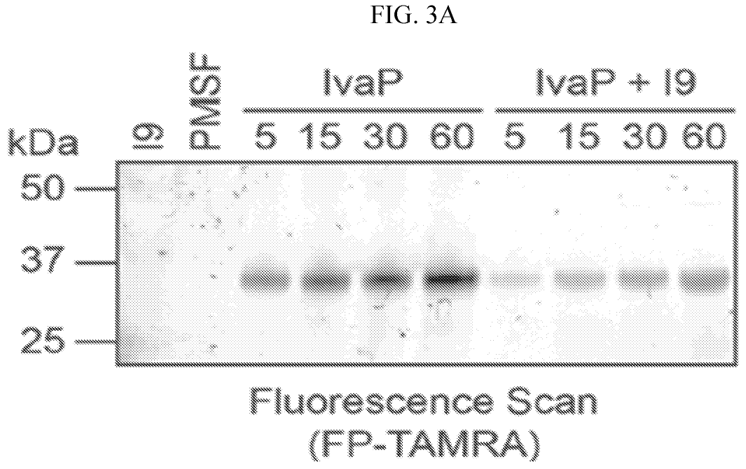

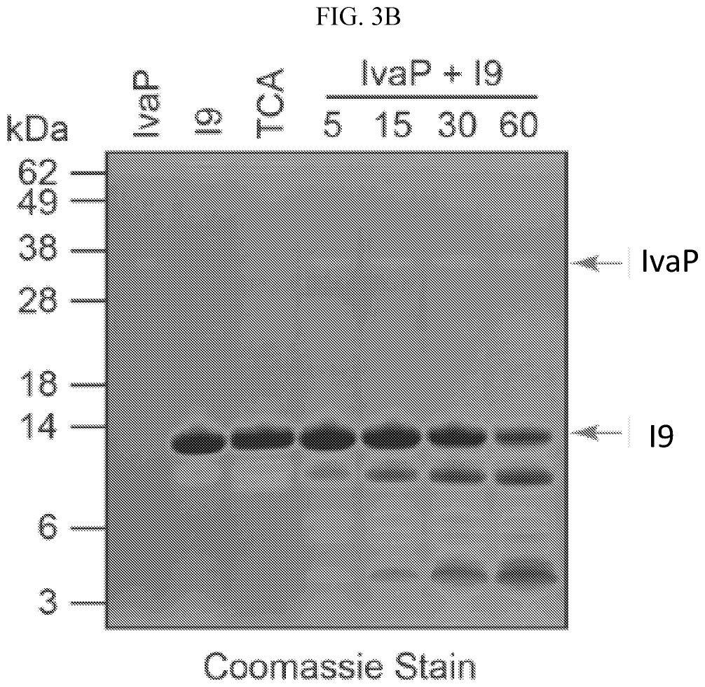

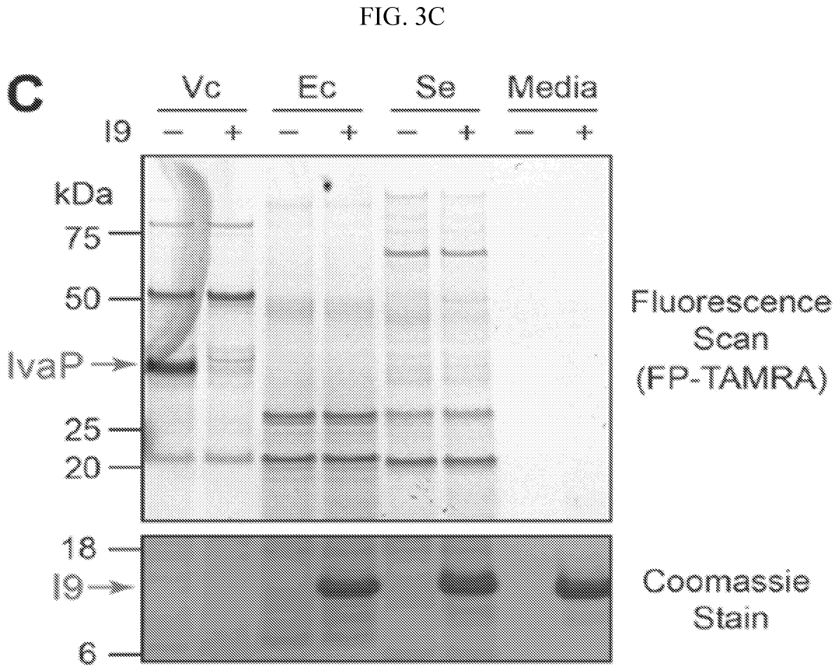

To identify propeptide-containing proteases that are active during intestinal infections, activity-based proteomics, a technique that uses chemical probes to selectively detect active enzymes in complex samples, can be used. Using a fluorophosphonate-based probe for serine hydrolases a serine protease, IvaP, which is secreted by the cholera pathogen Vibrio cholerae in the rabbit intestine and in human choleric stool, was identified. IvaP contains a propeptide from the I9 family of peptidase inhibitors that is cleaved extracellularly to generate the active enzyme. Purified I9 inhibitors can temporarily inhibit, and be degraded by, their cognate proteases in trans. Similarly, the IvaP propeptide is a temporary inhibitor (FIG. 3A) and substrate (FIG. 3B) of IvaP.

Furthermore, propeptide addition specifically inhibits IvaP activity and not the activity of other secreted serine hydrolases produced by cultures of V. cholerae or other intestinal bacteria (FIG. 3C, top panel). In addition, the IvaP propeptide is selectively degraded by V. cholerae culture supernatants (FIG. 3C, bottom panel). The IvaP I9 domain can be used as an illustrative propeptide for the development of a biosensor and/or and caged antimicrobial targeting V. cholerae. As a paradigmatic intestinal pathogen with robust in vivo infection models, V. cholerae is a viable target for the present studies. Similar design principles can be used to expand these technologies to other clinically important pathogens.

### Example 2: Selective Detection of Pathogen Proteases for Disease Diagnostics

The development of low-cost, rapid diagnostic tests for pathogens in complex samples is a major goal of infectious disease research. In particular, diagnostics that can be used directly on clinical isolates without extensive sample processing are highly desirable for point-of-care applications. It is estimated that such laboratory-independent tests could prevent over one million deaths each year. Fluorogenic biosensors, which become fluorescent only after engaging with the targeted analyte, can provide processing-free detection of whole bacteria by eliminating the wash-out steps required of fluorescent probes.

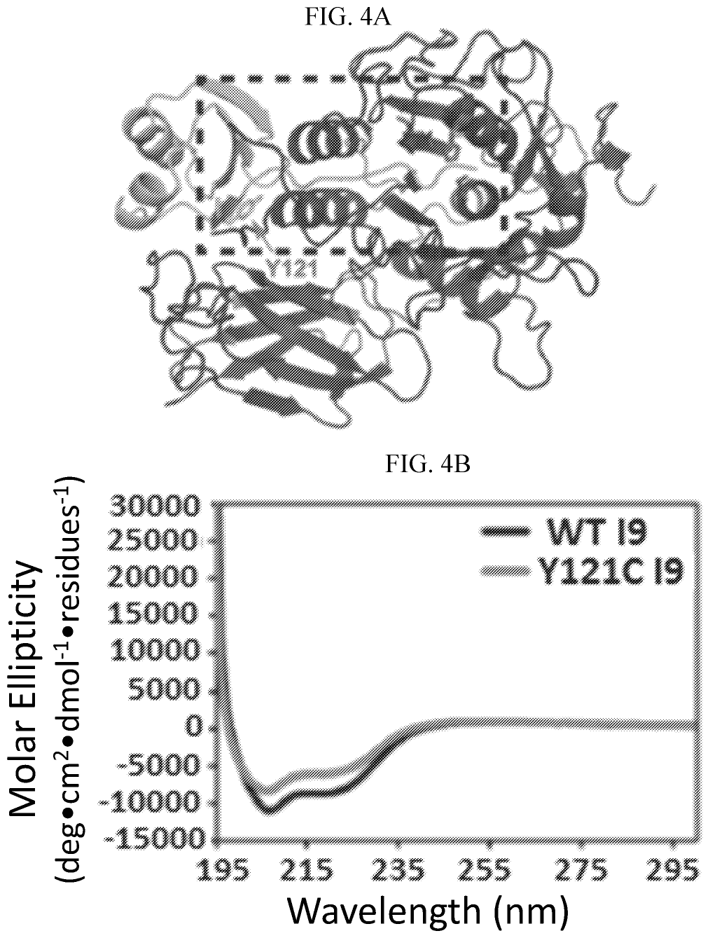

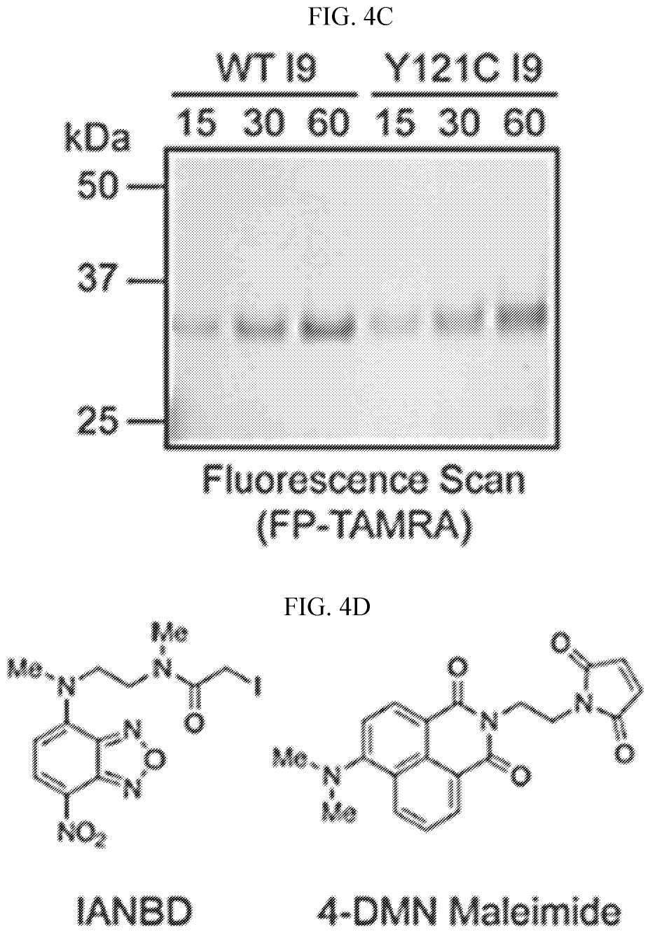

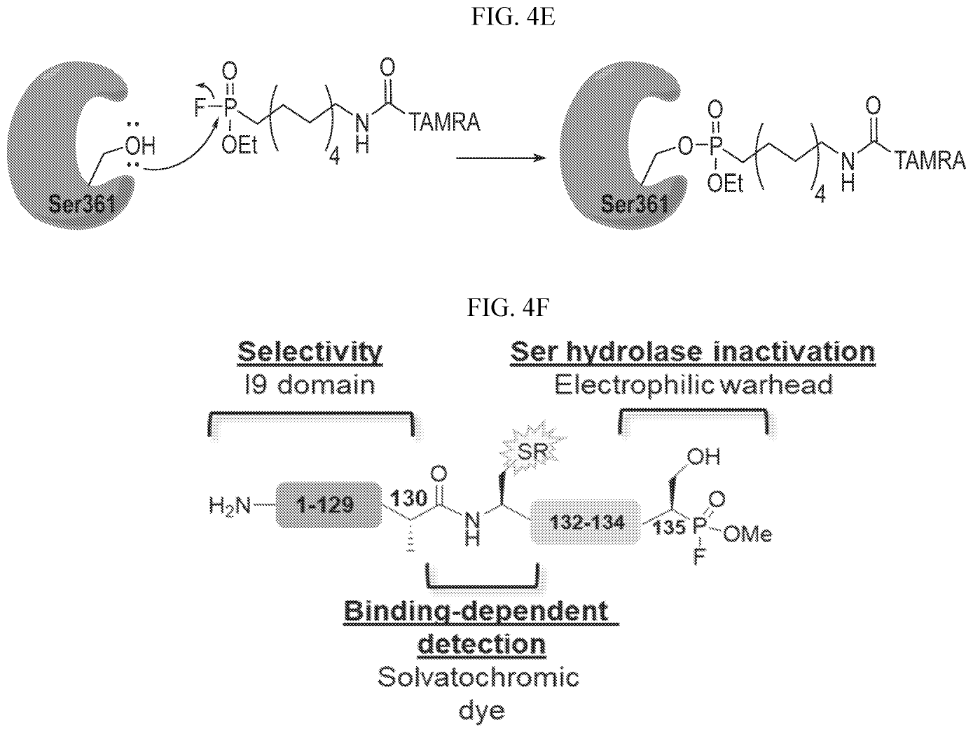

Protease-propeptide interaction (such as, in a non-limiting example, IvaP-I9) can be used to selectively detect a pathogenic microorganism (such as, but not limited to, V. cholerae) using environment-sensitive dyes that emit light in hydrophobic environments and are activated by protease binding. In a non-limiting example, I9 mutants containing cysteine residues are generated along the hydrophobic interface of the I9-IvaP interaction complex (FIG. 4A). Because the IvaP propeptide, like most I9 domains, does not contain any cysteines, mutagenesis provides a selective handle for bioconjugation. The tested cysteine substitution along the IvaP-binding surface of I9 did not significantly alter peptide structure (FIG. 4B) or protease binding (FIG. 4C). I9 mutants are conjugated with solvatochromic dyes (FIG. 4D), which exhibit up to a 700-fold increase in fluorescence intensity upon transition from aqueous to hydrophobic environments. Both commercial dyes and more sensitive variants are synthesized using established protocols (Loving & Imperiali, 2009, Bioconjug Chem 20:2133-2141). Probe binding to IvaP is monitored in vitro, in intestinal fluid, and in stool using a plate reader and fluorescence microscope. Given the specificity of I9 binding (FIG. 3C), probe addition should enable the no-wash fluorescence detection of IvaP. In certain embodiments, these studies generate sensitive biosensors for precise monitoring of a V. cholerae protease without requiring the design and synthesis of protease-specific substrates.

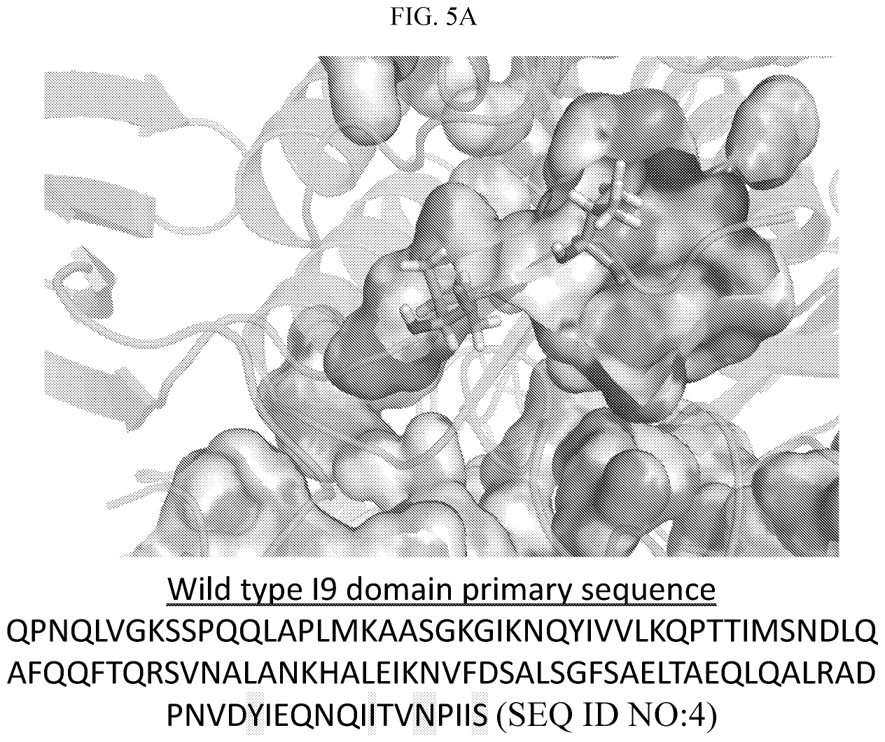

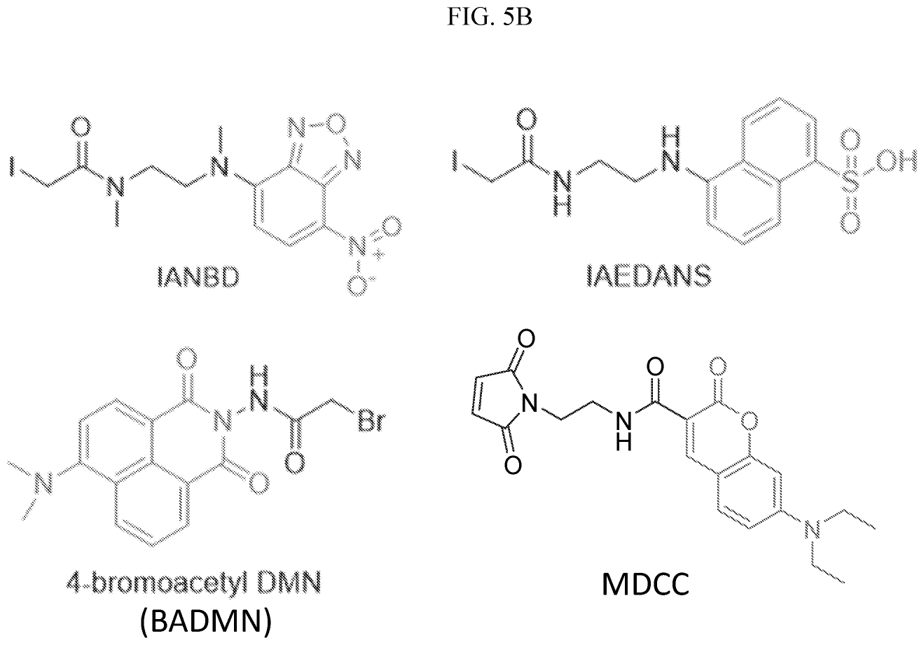

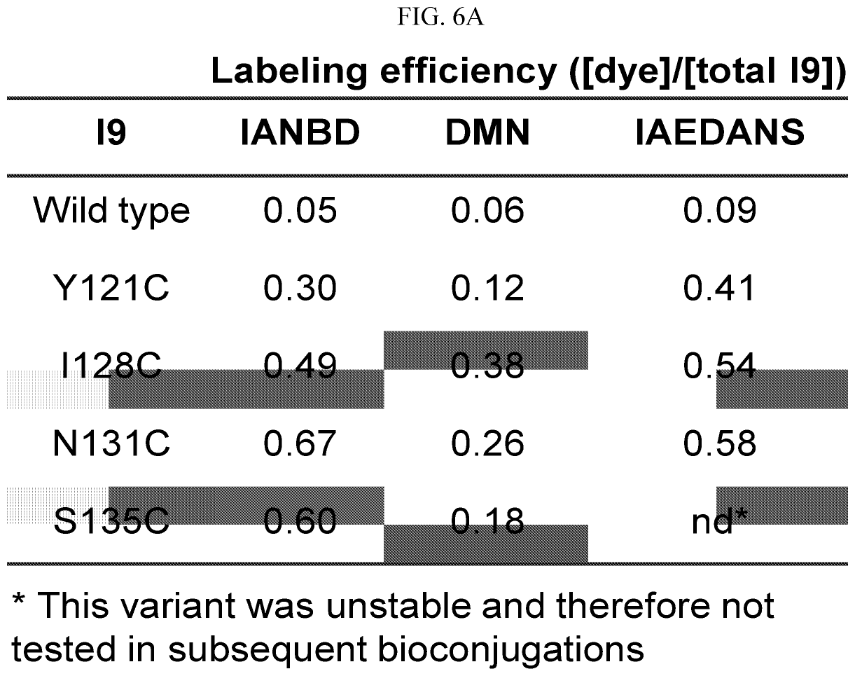

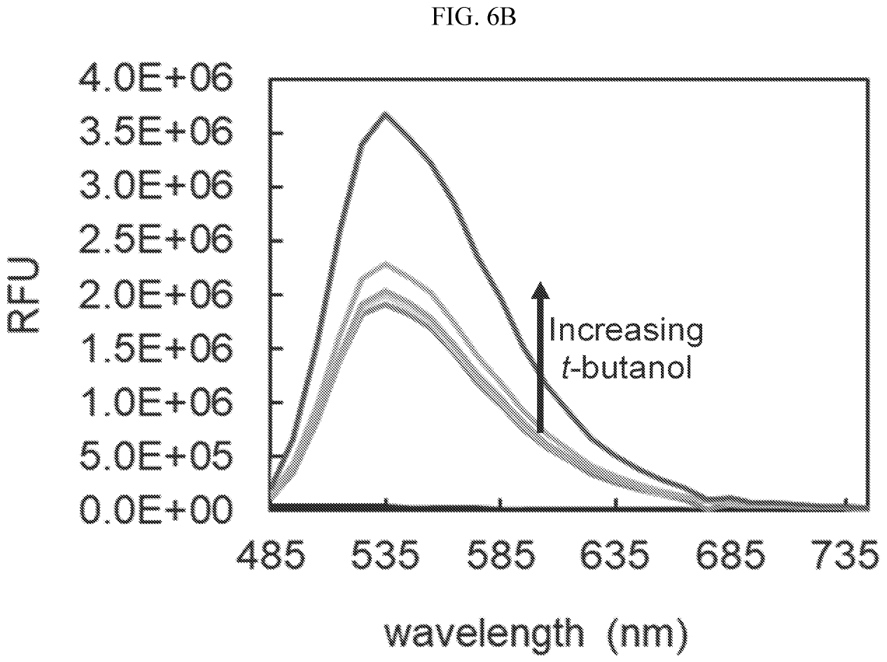

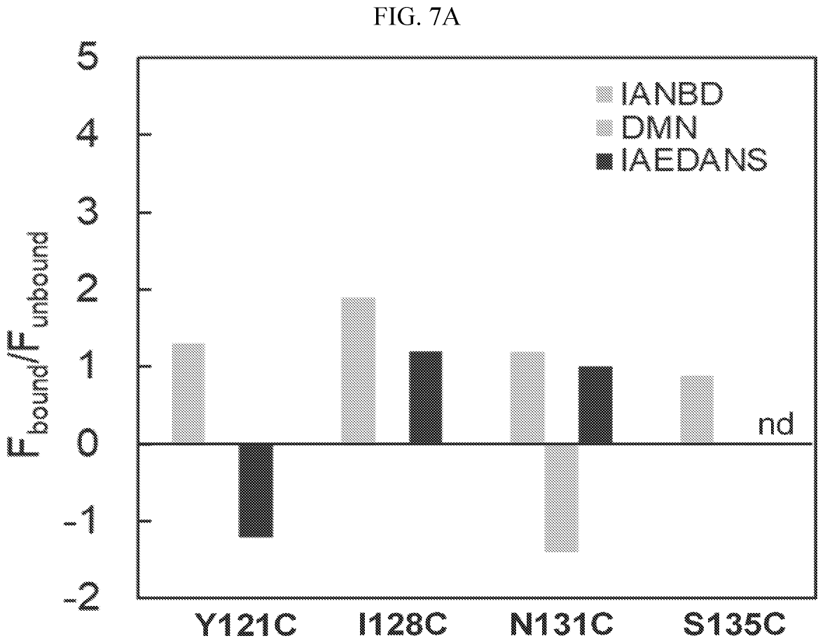

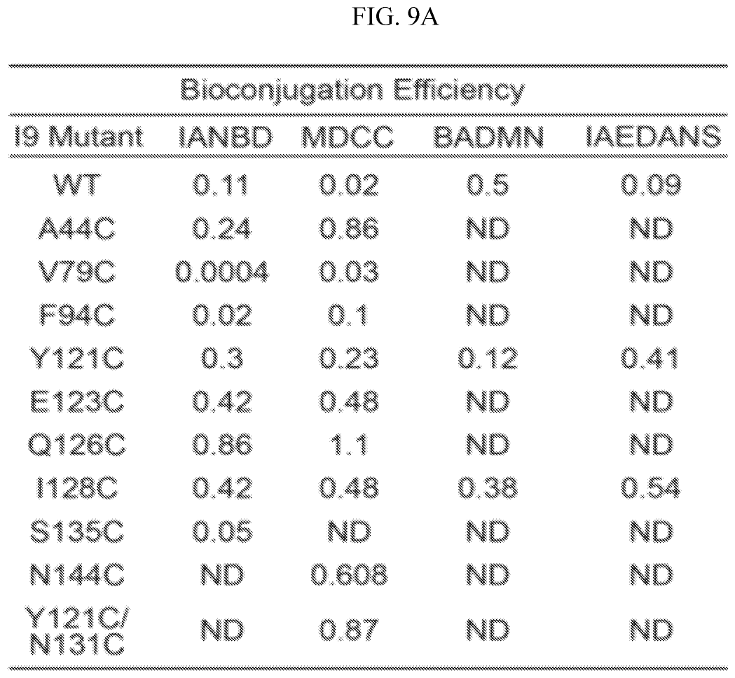

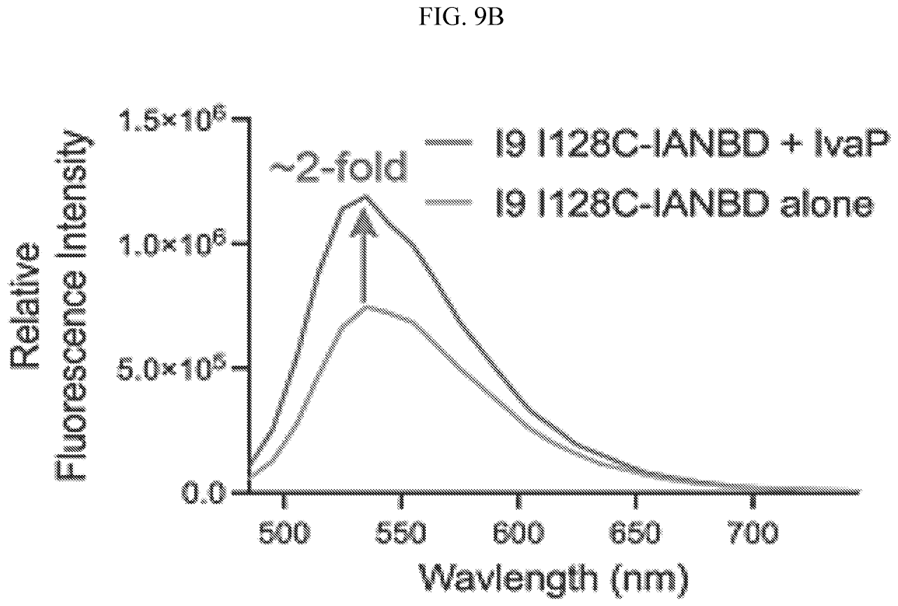

A series of I9 mutants containing cysteine residues along the predicted I9-IvaP binding interface was prepared and conjugated to a panel of thiol-reactive, environment-sensitive dyes (FIG. 5B). For each mutant, it was confirmed that cysteine substitution did not significantly alter the overall structure of I9 or its binding to IvaP using circular dichroism spectroscopy (not shown) and the FP-TAMRA competition assay (FIG. 3A), respectively. The efficiency of bioconjugation of each I9 mutant-dye pair was also compared to that of the corresponding WT I9-dye control to identify mutants for which dye labeling was enhanced by cysteine substitution (FIG. 9A).

Using the initial set of 21 I9-dye conjugates (aka probes), which consisted of 10 I9 mutants each conjugated to between one and four commercial or synthetized solvatochromic dyes, the fluorescence intensity of probe binding to IvaP was monitored in a multi-well plate assay using a fluorescence plate reader. A modest (˜2-fold) increase in fluorescence intensity was detected on incubating the I9 I128C-IANBD conjugate with purified IvaP in solution relative to the fluorescence intensity of the I9 I128C-IANBD conjugate alone (FIG. 9B). Although not wishing to be limited by theory, these results suggested that the I9-IvaP interface may not be sufficiently hydrophobic to elicit a significant increase in the fluorescence intensity of the tested solvatochromic dyes.

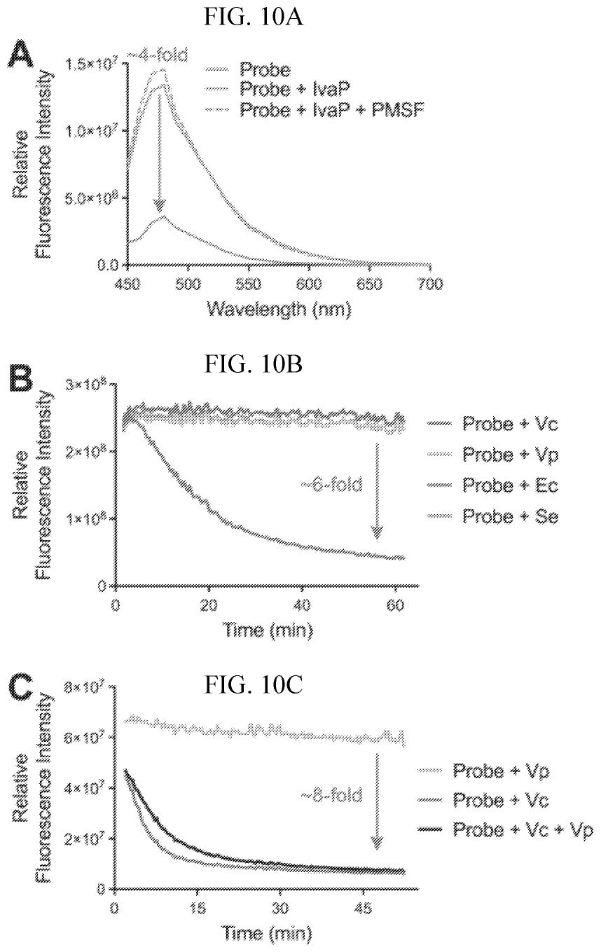

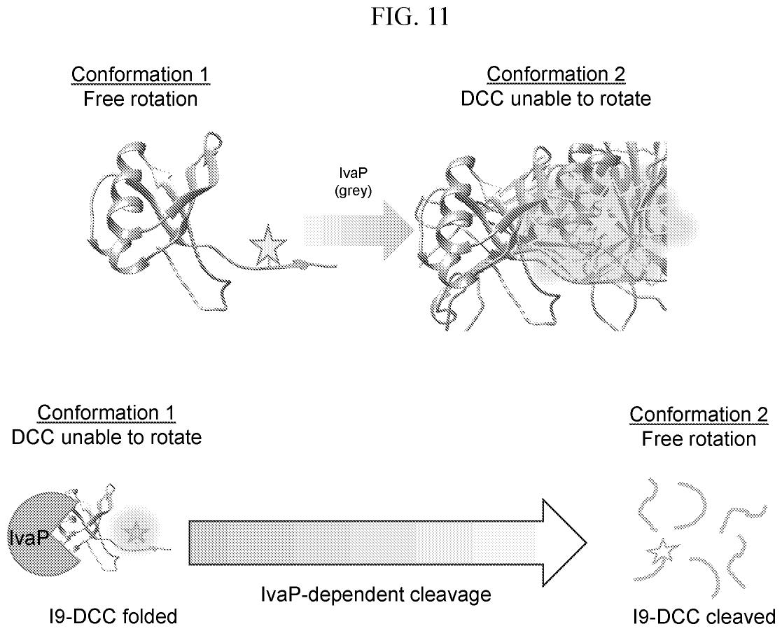

However, an I9-dye conjugate, I9 E123C-MDCC, was identified which unexpectedly exhibited a 4-fold decrease in fluorescence intensity following incubation with purified IvaP, but not IvaP pre-treated with the protease inhibitor PMSF (FIG. 10A). Unlike the other dyes included in the probe panel, whose optical properties are primarily determined by the extent of dye solvation, MDCC is a conformation-sensitive dye with a fluorescence intensity determined by the conformational state of the molecule: constrained rotation enhances MDCC fluorescence, whereas an increase in rotational flexibility decreases dye fluorescence (FIG. 11). Given that IvaP degrades I9 over time, the data suggest that the cleavage of I9 E123C-MDCC enhances the fluorescence of MDCC, most likely by cleaving the peptide backbone into smaller fragments that can diffuse and rotate more freely in solution (FIG. 11).

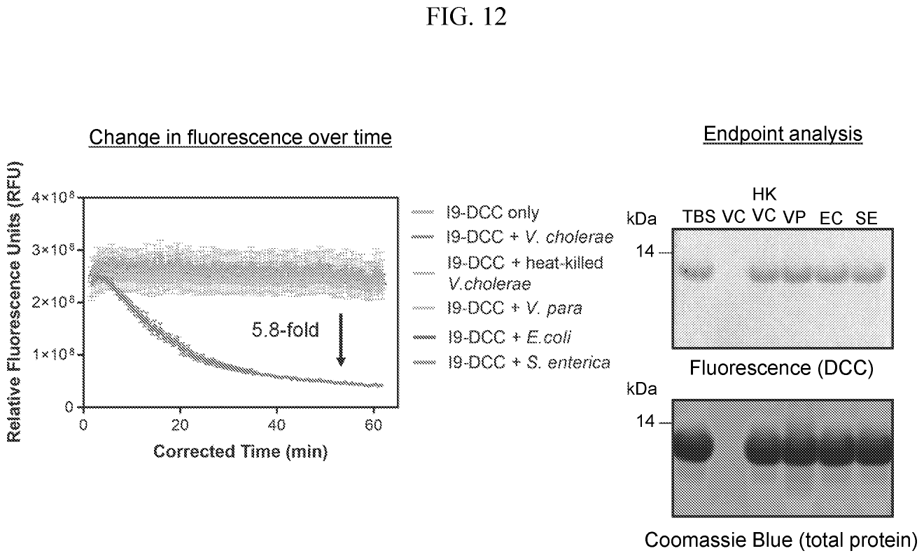

Given the significant IvaP-dependent decrease in fluorescence intensity of I9 E123C-MDCC, it was assessed whether this probe could be used to selectively detect V. cholerae bacteria in vitro. The probe was incubated with biofilm cultures of V. cholerae, S. enterica Typhimurium, E. coli, and Vibrio parahaemolyticus, another intestinal pathogen closely related to V. cholerae. Remarkably, a significant and specific decrease in the fluorescence intensity of the probe was detected in the presence of V. cholerae, but not any of the other tested bacteria (FIG. 10B). These results were validated by gel-based analyses using both fluorescence detection and Coomassie Blue staining (FIG. 12). In addition, it was determined that the probe can selectively detect V. cholerae grown in mixed cultures with V. parahaemolyticus; a selective, ˜8-fold decrease in probe intensity was observed only in the presence of V. cholerae (FIG. 10C). Together, these data support that the I9 E123C-MDCC probe is a selective sensor of V. cholerae in mixed cultures. In addition, they confirm that propeptide-based sensors are a viable approach for the selective detection of bacterial pathogens.

### Example 3: Protease-Activated Antimicrobials for Selectively Killing Pathogens in the Gut

Despite the enormous benefits of broad-spectrum antibiotics, their widespread use over the past century has resulted in a global healthcare crisis. The development of new antibiotics has not kept pace with the alarming rise of multidrug-resistant bacteria. By current estimates, unless new strategies are developed to inhibit the spread of antibiotic resistance, the annual toll of drug-resistant infections will reach ˜10 million deaths by the year 2050. Precision antimicrobials-antimicrobials that target specific pathogens within complex microbial communities-promise to significantly reduce the threat of drug resistance and chronic illness associated with off-target killing of the microbiota.

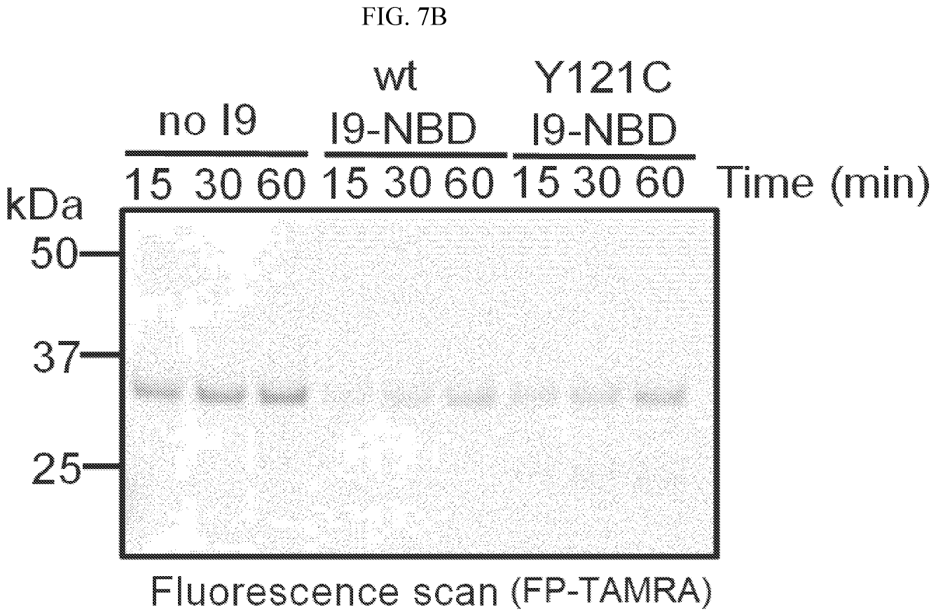

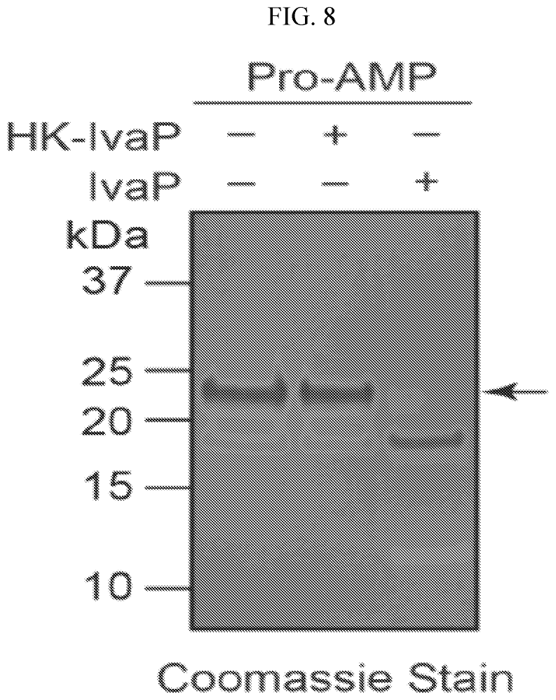

The present disclosure provides a new class of precision antimicrobials that are activated by propeptide cleavage. In certain embodiments, propeptides from pathogen proteases are fused to C-terminal antimicrobial peptides (AMPs), potent antibiotics that punch holes in bacterial membranes. Because N-terminal fusions inhibit AMP toxicity, the propeptide-bound AMP (i.e., prodrug) remains inert. Propeptide cleavage releases the AMP in the vicinity of the protease-producing pathogen, increasing its concentration at the pathogen cell surface. Since most commensal microbes in the human gut are naturally resistant to AMPs, AMP release should not significantly impact the microbiota. As a non-limiting example, a Vibrio-toxic AMP fused to an N-terminal propeptide sequence was successfully purified and shown to be uncaged by IvaP (FIG. 8). In certain embodiments, the present disclosure includes prodrugs targeting surface-associated proteases. The IvaP-dependent killing of V. cholerae by these caged AMPs can be evaluated in vitro and in vivo. In parallel, the specificity of prodrug cleavage can be assessed using appropriate controls, including IvaP-deficient strains of V. cholerae and cultures of other intestinal bacteria. The well-defined structure of propeptides from the I9 family (FIGS. 4A-4B) and the high specificity of propeptide-protease interactions (FIG. 3C) should minimize nonspecific AMP activation by other proteases.

Although not wishing to be limited by theory, the data in Example 2 suggest that the folded structure of the full-length I9 propeptide is important for selectivity, however the construct in FIG. 8 contains an abbreviated I9 sequence. Given the initial success of the cleavage-activated I9 E123C-MDCC sensor, which is selectively degraded by V. cholerae in vitro (FIGS. 10B and 10C), a new panel of I9-AMP fusion constructs was cloned that include the full-length I9 sequence. This panel includes constructs for both the cytosolic and extracellular localization of each fusion protein in E. coli and V. cholerae. The initial studies suggest that expression of the cytosolic constructs is toxic; thus, secreted constructs are being purified using techniques previously optimized for IvaP purification. In addition, the well-characterized, cationic AMP LL-37 is being used as the initial model payload since commercial antibodies can be readily used to validate prodrug expression and to monitor LL37 cleavage and localization. Once a panel of I9-AMP prodrugs has been synthesized, the IvaP-dependent killing of V. cholerae by these caged AMPs will be evaluated in vitro and in vivo. In parallel, the specificity of prodrug cleavage will be assessed using appropriate controls, including IvaP-deficient strains of V. cholerae and cultures of other intestinal bacteria.

### Example 4: Propeptide-Based Biosensors and Prodrugs Targeting C. difficile

C. difficile is the most common cause of hospital-acquired infections in the United States. C. difficile infection (CDI) is characterized by the development of life-threatening intestinal inflammation that claims ˜30,000 lives annually. Because broad-spectrum antibiotics exacerbate the symptoms of CDI, alternative therapies that selectively kill C. difficile are desperately needed. Furthermore, because C. difficile does not grow under standard laboratory conditions, fecal samples cannot be rapidly screened using culture-based diagnostics. These challenges can be addressed by adapting the present propeptide-based technology to target proteases from C. difficile. Activity-based proteomics is used to identify propeptide-containing proteases that are active in fecal samples from C. difficile-infected mice and humans. In parallel, the technology is applied to the I9 propeptide of CspB, a C. difficile protease that is required for the germination of infectious C. difficile spores. Because spore germination occurs in the intestine at the onset of CDI, CspB activity can provide an early marker of infection and can potentially be targeted to prevent the development of disease.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A construct comprising:
a pathogenic bacterium protease propeptide conjugated to a detectable label, an antimicrobial peptide (AMP), or a therapeutically active small molecule;
wherein the detectable label is a fluorophore comprising a solvatochromic label, a chromophore, or a magnetic nanoparticle;
wherein the detectable label emits a detectable signal once the propeptide is cleaved; and
wherein the detectable label is attached to:
(i) a residue of the propeptide that is present in the interface of a propeptide-protease complex once the propeptide binds to its corresponding protease; or
(ii) a residue of the propeptide that is at a protease cleavage site;
wherein the solvatochromic label comprises one of the following: 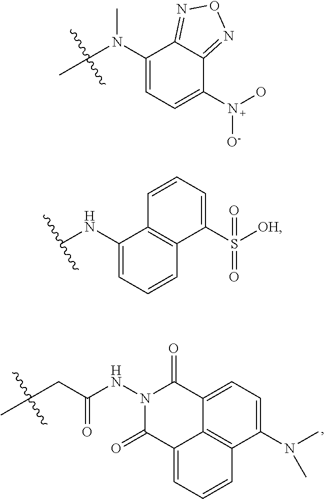 [SMILES: *N(C)c1ccc(N([O-])O)c2nonc12 ; *Nc1cccc2c(S(=O)(=O)O)cccc12 ; *CC(=O)NN1C(=O)c2cccc3c(N(C)C)ccc(c23)C1=O]
a charge transfer dye, an excited-state intramolecular proton transfer dye, or a merocyanine dye.

2. (canceled)

3. (canceled)

4. (canceled)

5. The construct of claim 1, wherein at least one of the following applies:
i) the fluorophore comprises a TCC probe;
ii) the fluorophore is a conformation-sensitive fluorophore; and
iii) the fluorophore is a conformation-sensitive fluorophore comprising the structure

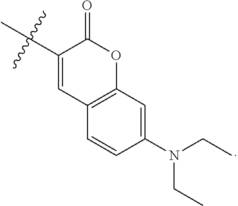 [SMILES: *c1cc2ccc(N(CC)CC)cc2oc1=O]

6.-9. (canceled)

10. The construct of claim 1, wherein at least one of the following applies:
i) the propeptide is conjugated to the detectable label, the AMP, or the therapeutically active small molecule through a direct covalent bond;
ii) the propeptide is conjugated to the N-terminus of the AMP;
iii) the propeptide is conjugated to the detectable label, the AMP, or the therapeutically active small molecule through a linker;
iv) the propeptide is conjugated to the detectable label, the AMP, or the therapeutically active small molecule through a linker of about 1 to about 100 amino acids; and
v) the propeptide is conjugated to the detectable label, the AMP, or the therapeutically active small molecule through a linker of about 1 to about 50 residues selected from the group consisting of CH₂, CH₂CH₂, —C(═O)NH—, —C(═O)NCH₃—, CH(CH₃)CH₂, CH₂CH₂O, C(CH₃)CH₂O, and

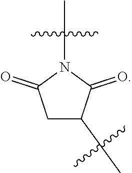 [SMILES: *C1CC(=O)N(*)C1=O]

11. (canceled)

12. (canceled)

13. The construct of claim 10, wherein the linker is

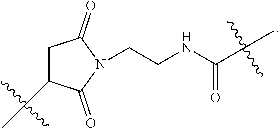 [SMILES: *C(=O)NCCN1C(=O)CC(*)C1=O]

14. The construct of claim 1, wherein the protease is subtilisin.

15. The construct of claim 1, wherein one of the following applies:
i) the propeptide is a I9 propeptide;
ii) the propeptide is a I9 propeptide for IvaP from Vibrio cholerae or a I9 propeptide for CspB of C. difficile;
iii) the propeptide is from Protease IV; and
iv) the propeptide is from Protease IV from Pseudomonas aeruginosa.

16.-30. (canceled)

31. A construct comprising a pathogenic bacterium protease propeptide conjugated to an electrophilic agent.

32. The construct of claim 31, wherein the electrophilic agent is conjugated to the C-terminus of the propeptide or a mutated residue of the propeptide.

33. The construct of claim 31, wherein the electrophilic agent comprises a fluorophosphate group.

34. The construct of claim 31, wherein at least one of the following applies:
i) the propeptide is conjugated to the electrophilic agent through a direct covalent bond;
ii) the propeptide is conjugated to the electrophilic agent through a linker;
iii) the propeptide is conjugated to the electrophilic agent through a linker of about 1 to about 100 amino acids; and
iv) the propeptide is conjugated to the electrophilic agent through a linker of about 1 to about 50 residues selected from the group consisting of CH₂, CH₂CH₂, —C(═O)NH—, —C(═O)NCH₃—, CH(CH₃)CH₂, CH₂CH₂O, C(CH₃)CH₂O, and

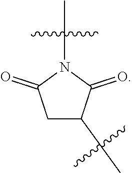 [SMILES: *C1CC(=O)N(*)C1=O]

35. (canceled)

36. (canceled)

37. The construct of claim 31, wherein the protease is a subtilisin.

38. The construct of claim 31, wherein one of the following applies:
i) the propeptide is a I9 propeptide;
ii) the propeptide is a I9 propeptide for IvaP from Vibrio cholerae or a I9 propeptide for CspB of C. difficile;
iii) the propeptide is from Protease IV; and
iv) the propeptide is from Protease IV from Pseudomonas aeruginosa.

39-41. (canceled)

42. A method of identifying a pathogenic bacterium in a sample, the method comprising:
contacting the construct of claim 1 with the sample, wherein the bacterium produces the protease that binds to the propeptide, and
monitoring a detectable signal for the detectable label before and after the contacting,
whereby a qualitative or quantitative change in the detectable signal upon contacting indicates that the pathogenic bacterium is present in the sample.

43. The method of claim 42, wherein the sample comprises another bacterium that does not produce the protease that binds to the propeptide.

44. The method of any claim 42, wherein the detectable label comprises a fluorophore, chromophore, or any other detectable label.

45. A method of killing a pathogenic bacterium, or reducing or preventing growth of a pathogenic bacterium, the method comprising:
contacting a sample containing at least one pathogenic bacterium and at least one non-pathogenic bacterium with the construct of claim 1,
wherein the pathogenic bacterium produces the protease that binds to the propeptide; and
whereby the AMP is released from the construct upon the contacting.

46. The method of claim 45, wherein the method is performed in vivo in a subject infected by the pathogenic bacterium.

47. The method of claim 46, wherein the pathogenic bacterium is in the digestive tract, respiratory tract, urinary tract, or skin of the subject.

48. The method of claim 46, wherein non-pathogenic bacteria in the gut of the subject are not significantly killed, or wherein growth of non-pathogenic bacteria in the gut is not significantly reduced or prevented.

49. A method of inactivating a protease of a pathogenic bacterium, the method comprising:
contacting the construct of claim 31 with the pathogenic bacterium,
wherein the pathogenic bacterium produces the protease that binds to the propeptide; and
wherein the electrophilic agent is conjugated to the pathogenic bacterium protease propeptide.


SEQUENCE LISTING (4 sequences)

SEQ ID NO: 1 - protein, 535 aa, Artificial Sequence
  Chemically Synthesized
MFKKFLSLCIVSTFSVAATSALAQPNQLVGKSSPQQLAPLMKAASGKGIKNQYIVVLKQPTTIMSNDLQAFQQFTQRSVNALANKHALEIKNVFDSALSGFSAELTAEQLRGIDWVAQNASGPSVANLSLGGGISQAMDQAVARLVQRGVTAVIAAGNDNQALRADPNVDYIEQNQIITVNPIISASANAAQDNVTWGIDRIDQRDLPLNKDACQVSPAREPSGITVGSTTNNDGRSNFSNWGNCVQIFAPGSDVTSASHRSYNYNYDGSGVTAYVIDTGIAFNHPEFGGRAKSGYDFIDNDNDASDCQGHGTHVAGTIGKGGTTTMSGTSMASPHVAGVAALYLQENKNLSPNQIKTLLSDRSTKGKVSGAQYGVAKNVNLVGVRVLGCDGSGSTEAIADTQGTPNKLLYSLTDNNTTPNPEPNPQPEPQPQPDSQLTNGKVVTGISGKQGELKKFYIDVPAGRRLSIETNGGTGNLDLYVRLGIEPEPFAWDCASYRNGNNEVCTFPNTREGRHFITLYGTTEFNNVSLVARY

SEQ ID NO: 2 - protein, 548 aa, Artificial Sequence
  Chemically Synthesized
MIIINYELIVKYNGDILRLEEELGVSVEILNSSYAIITSSNEEDVNILLTYPEIEFIEKPFILQTQDVQSFSSTGITGFKNRTGLTGKGTIIGIIDSGIDYTLPVFRDSDGRSKILYYWDQSIQGNPPEGFREGTLYTNEDINNAIDGSMYIPISTTSLHGTHVAGICATIASDARIIVVRVGNIQTDIFSRSTEFMRAIKFILDRALELRMPVTLNISYGSNEGSHRGTSLFEQYIDDMCLFWKNNIVVAAGNNADKGGHKRIRLQNNITEEVEFIVGEGERILNINIWPDFVDDFSVHLVNPSNNQTQAISLTSGEIRNTLGETRITGYFYPIAPYSLTRRVTLQLSSNTQITPGLWKIVFEPIDIVTGNVNIYLPTSEGLNRNTRFLIPTQELTVTVPGTASRVITVGSFNSRTDIVSIFSGEGDTQLGVFKPDLLAPGEDIVSFLPGGTSGALTGTSMATPHVTGVCSLFMEWGIVNGNDLFLYSQKLRALLLKGARRLSNQSYPNNSSGFGFLNLSDIDLYTLSNINQDLETEDMGYRSINKS

SEQ ID NO: 3 - protein, 462 aa, Artificial Sequence
  Chemically Synthesized
MHKRTYLNACLVLALAAGASQALAAPGASEMAGDVAVLQASPASTGHARFANPNAAISAAGIHFAAPPARRVARAAPLAPKPGTPLQVGVGLKTATPEIDLATLEWIDTPDGRHTARFPISAAGAASLRAAIRLETRSGSLPDDVLLHFAGDGKEIFEASGKDLSVNRPYWSPVIEGDTLTVELVLPANLQPGDLRLSVPQVSYFADSLYKAGYRDGFGASGSCEVDAVCATQSGTRAYDNATAAVAKMVFTNSADGGSYICTGTLLNNGNSPKRQLFWSAAHCIEDQATAATLQTIWFYNTTQCYGDASTINQSVTVLTGGANILHRDAKRDTLLLELKRTPPAGVFYQGWSATPIANGSLGHDIHHPRGDAKKYSQGNVSAVGVTYDGHTALTRVDWPSAVVEGGSSGSGLLTVAGDGSYQLRGGLYGGPSYCGAPTSQRNDYFSDFSGVYSQISRYFAP

SEQ ID NO: 4 - protein, 112 aa, Artificial Sequence
  Chemically Synthesized
  MISC_FEATURE (98)..(98): Xaa = Tyr or Cys
  MISC_FEATURE (105)..(105): Xaa = Ile or Cys
  MISC_FEATURE (108)..(108): Xaa = Asn or Cys
  MISC_FEATURE (112)..(112): Xaa = Ser or Cys
QPNQLVGKSSPQQLAPLMKAASGKGIKNQYIVVLKQPTTIMSNDLQAFQQFTQRSVNALANKHALEIKNVFDSALSGFSAELTAEQLQALRADPNVDXIEQNQIXTVXPIIX